SEQUENCE LISTING (13 sequences)

SEQ ID NO: 1 - protein, 170 aa, Human
ASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 2 - protein, 13 aa, Artificial Sequence
  synthetic
VHFFKNIVTPRTP

SEQ ID NO: 3 - protein, 13 aa, Artificial Sequence
  synthetic
VHFFGNIVTPRTP

SEQ ID NO: 4 - protein, 13 aa, Artificial Sequence
  synthetic
VHFFANIVTPRTP

SEQ ID NO: 5 - protein, 13 aa, Artificial Sequence
  synthetic
VHFFKNIVTARTP

SEQ ID NO: 6 - DNA, 21 bp, Artificial Sequence
  synthetic
ctgaaggtcaaagggaatgtg

SEQ ID NO: 7 - DNA, 21 bp, Artificial Sequence
  synthetic
ggacagagtcttgatgatctc

SEQ ID NO: 8 - DNA, 19 bp, Artificial Sequence
  synthetic
actgccttccctacttcac

SEQ ID NO: 9 - DNA, 21 bp, Artificial Sequence
  synthetic
gtattgctctgaatgactctg

SEQ ID NO: 10 - DNA, 25 bp, Artificial Sequence
  synthetic
aggaggcgctccccaaaaagatggg

SEQ ID NO: 11 - DNA, 22 bp, Artificial Sequence
  synthetic
gtacatgggctcataccagttg

SEQ ID NO: 12 - DNA, 20 bp, Artificial Sequence
  synthetic
agatgacatcacctggacct

SEQ ID NO: 13 - DNA, 21 bp, Artificial Sequence
  synthetic
ctttggttcagtgtgaccttc

(19) United States
(12) Patent Application Publication
(10) Pub. No.: US 2006/0057110 A1
(43) Pub. Date: Mar. 16, 2006

(54) Antigen-presenting cells for neuroprotection and nerve regeneration

(75) Inventors: Michal Eisenbach-Schwartz, Rehovot (IL); Avraham Cohen, Givataim (IL)

Correspondence Address: BROWDY AND NEIMARK, P.L.L.C.;624 NINTH STREET, NW, SUITE 300, WASHINGTON, DC, 20001-5303, US

(73) Assignee: Yeda Research and Development Co. Ltd., Rehovot (IL)

(21) Appl. No.: 10/517,666

(22) PCT Filed: Jun. 12, 2003
(86) PCT No.: PCT/IL03/00500

§ 371 Date: Dec. 13, 2004

Related U.S. Application Data
(60) Provisional application No. 60/388,296, filed on Jun. 14, 2002.

(51) Int. Cl. A61K 38/17; A61K 35/14

(52) U.S. Cl. 424/93.1; 514/12

(57) ABSTRACT

Pharmaceutical compositions and methods for preventing or inhibiting neuronal degeneration, or for promoting nerve regeneration, in the central nervous system (CNS) or peripheral nervous system (PNS), in the treatment of an injury, disorder or disease of the CNS or PNS, comprise antigen-presenting cells, preferably dendritic cells, that have been pulsed with an agent selected from the group consisting of: (a) a nervous system (NS)-specific antigen or an analog thereof; (b) a peptide derived from an NS-specific antigen or from an analog thereof, or an analog or derivative of said peptide; (c) a copolymer selected from the group consisting of Copolymer 1, a Copolymer 1-related peptide or polypeptide, and poly-Glu<50> Tyr<50>; and (d) a non-self antigen.

### FIELD OF THE INVENTION

The present invention relates to compositions and methods and, more particularly, to compositions comprising antigen-presenting cells, preferably dendritic cells, pulsed with a suitable antigen, and to the use of said antigen-pulsed cells in methods for preventing or inhibiting neuronal degeneration or for promoting nerve regeneration in the central nervous system (CNS) or peripheral nervous system (PNS).

Abbreviations: APC: antigen-presenting cells; APL: altered peptide ligand; CNS: central nervous system; BBB: Basso, Beaffie and Bresnahan open-field locomotion scale; DC: dendritic cells; EAE: experimental autoimmune encephalomyelitis; GM-CSF: granulocyte-macrophage colony-stimulating factor; MBP: myelin basic protein; MHC: major histocompatibility complex; NS: nerve system; PNS: peripheral nervous system; RT-PCR: reverse transcription-polymerase chain reaction; SCI: spinal cord injury.

### BACKGROUND OF THE INVENTION

The nervous system comprises the central nervous system (CNS), composed of the brain and spinal cord, and the peripheral nervous system (PNS), consisting of the nerves and ganglia outside the brain and spinal cord. Damage to the nervous system may result from a traumatic injury, such as penetrating trauma or blunt trauma, or a disease or disorder including Alzheimer's disease, Parkinson's disease, multiple sclerosis, Huntington's disease, amyotrophic lateral sclerosis (ALS), diabetic neuropathy, senile dementia, and ischemia.

While the immune system plays an essential part in protection, repair, and healing in most tissues, immunological reactions are relatively limited in the CNS, due to its unique immune privilege. The failure of the mammalian CNS to achieve functional recovery after injury reflects an ineffective dialog between the damaged tissue and the immune system. Thus, the restricted communication between the CNS and blood-borne macrophages affects the capacity of axotomized axons to regrow, but transplants of activated macrophages have been shown to promote CNS axonal regrowth (Rapalino et al., 1998).

Since neurons in the mammalian CNS do not undergo spontaneous regeneration following an injury, a CNS injury may often lead to permanent impairment of motor and sensory functions.

Spinal cord injury (SCI) often has a devastating outcome, which results not only from damage to directly injured neurons and poor regeneration, but also from secondary damage to neighboring neurons that escaped the initial injury. These secondary events are caused mainly by the activity of injury-evoked destructive self-compounds, such as physiological substances in toxic excess of their normal levels or degradation products of self-compounds (Faden, 1993). Recovery from SCI may thus be improved by preventing the spread of damage (i.e., by neuroprotection) and by promoting regrowth of damaged fibers whose cell bodies are still viable (i.e. by regeneration) (Basso, et al., 1996; Bavetta, et al., 1999; Bazan, et al., 1995; Beattie, et al., 1997; Behrmann, et al., 1994; Bethea, et al., 1999; Blesch and Tuszynski, 1997; Bregman, 1998; Brewer, et al., 1999; Constantini and Young, 1994; Crowe, et al., 1997; Franzen, et al., 1998; Hauben, et al., 2000; Liu, et al., 1994; Moalem, et al., 1999).

Immune involvement in CNS injuries has long been thought to have a deleterious effect on recovery and to play an active part in mediating secondary damage. The primary lesion causes changes that include local inflammation. Until very recently, the consensus was that inflammatory cells are responsible, in part, for the spread of damage and, accordingly, that any immune activity after spinal cord injury should be avoided or minimized. Attempts were therefore focused on reducing the inflammation after spinal cord injury by treatment with large doses of anti-inflammatory agents such as methylprednisolone.

Studies over the last few years have provided evidence indicating that cell-mediated immunity, if properly controlled, plays a pivotal role in regrowth of the injured spinal cord and its protection from secondary degeneration (Butovsky, et al., 2001; Hauben, et al., 2001; Hauben, et al., 2000; Hauben, et al., 2001; Hauben, et al., 2000). A properly controlled immune response after spinal cord or optic nerve injury helps to protect spared fibers from secondary degeneration, rescue cell bodies of damaged fibers, and promote regrowth of severed axons. Passive or active immunization with T cells specific to central nervous system (CNS)-associated myelin antigens reduces secondary degeneration in rat and mouse models of optic nerve crush or spinal cord contusion (Fisher, et al., 2001; Hauben, et al., 2000; Moalem, et al., 1999; Yoles, et al., 2001). Moreover, local implantation of macrophages activated by an autologous sciatic nerve in a completely transected spinal cord or optic nerve leads to regenerative growth with some recovery of function (Franzen, et al., 1998; Lazarov-Spiegler, et al., 1996; Rapalino, et al., 1998).

U.S. Pat. No. 5,800,812, No. 6,117,424 and No. 6,267,955, all assigned to present applicants, disclose methods and compositions for the use of allogeneic mononuclear phagocytes to promote axonal regeneration in the CNS of a mammal. Prior to administration into the CNS of the mammal at or near a site of injury, for example, in injured spinal cord, the mononuclear phagocytes are preferably activated by culturing them together with a stimulatory tissue such as a nerve segment, dermis, skin or medium conditioned by one or more of said stimulatory tissues, or with stimulatory cells or medium conditioned by stimulatory cells, or with at least one biologically active agent e.g. a cytokine such as GM-CSF, IL-2, IL-3, IL-4, IL-10.

PCT Publication No. WO 99/60021 of the present applicants describes compositions for preventing or inhibiting degeneration in the CNS or PNS for ameliorating the effects of injury or disease, comprising a nervous system (NS)-specific antigen such as myelin basic protein (MBP), a peptide derived therefrom or T cells activated therewith. PCT Publication No. WO 02/055010 of the present applicants discloses pharmaceutical compositions for promoting nerve regeneration or reducing or inhibiting degeneration in the CNS or PNS to ameliorate the effects of injury or disease comprising a peptide obtained by modification of a self-peptide derived from a CNS-specific antigen, which modification consists in the replacement of one or more amino acid residues of the self-peptide by different amino acid residues, said modified CNS peptide still being capable of recognizing the T-cell receptor recognized by the self-peptide but with less affinity, or T cells activated by such a modified CNS peptide.

The copolymer Cop 1 and T cells activated therewith were shown to confer neuroprotection and to protect CNS cells from glutamate toxicity. PCT Publications WO 01/52878 and WO 01/93893, both of the present applicants, disclose that Cop 1, Cop 1-related peptides and polypeptides and T cells activated therewith protect CNS cells from glutamate toxicity and prevent or inhibit neuronal degeneration or promote nerve regeneration in the CNS or PNS. WO 03/0022140, also of the present applicants, discloses that the copolymer poly-Glu⁵⁰Tyr⁵⁰ (formerly called polyGT and also designated polyYE) and T cells activated therewith protect CNS cells from glutamate toxicity and also prevent or inhibit neuronal degeneration or promote nerve regeneration in the CNS or PNS. Specifically, it was shown in said applications that in optic nerve fibers, the number of surviving retinal ganglion cells was significantly higher in the Cop 1-immunized or poly-Glu,Tyr-immunized mice than in the mice immunized with the adjuvant and PBS. Each and all patents and patent applications cited hereinabove are hereby incorporated by reference in their entirety as if fully disclosed herein.

An effective immune response involves two major groups of cells: lymphocytes (B and T cells) and antigen-presenting cells. Unlike membrane-bound antibodies on B cells, which can recognize antigen alone, T-cell receptors on the membrane can recognize only antigen that is bound to cell-membrane glycoproteins called major histocompatibility complex (MHC) molecules. There are two major types of MHC molecules: class I MHC molecules are expressed by nearly all nucleated cells, and class II MHC molecules are expressed only by antigen-presenting cells (APCs). T helper (T_{H}) cells, characterized by the presence of CD4 membrane glycoprotein on their surface, are activated when they recognize antigen that is displayed together with class MHC II molecules on the surface of APCs.

APCs first internalize antigen and then display a part of that antigen bound to a class II MHC molecule, on their membrane. The T_{H} cell recognizes and interacts with the antigen-class II MHC molecule complex on the membrane of the APC. An additional co-stimulatory signal is then produced by the APC, leading to the activation of the T_{H} cell.

A variety of cells can function as APCs. The distinguishing feature of these cells is their ability to express class II MHC molecules and to deliver a co-stimulatory signal. Three cell types are classified as professional APCs: dendritic cells, macrophages and B lymphocytes.

Dendritic cells (DCs) descend from hematopoietic stem cells through the myeloid lineage but the exact pathway of their development is not fully elucidated. It is not clear whether DCs develop as part of the monocyte/macrophage lineage or from an entirely separate lineage. Blood DCs develop from bone marrow myeloid precursors and then differentiate in the tissues into different types of DCs classified according to their tissue specific location, namely: Langerhans cells (epidermis and mucous membrane), and interstitial (heart, lungs, liver, kidney, gastrointestinal tract), interdigitating (present in T-cell areas of secondary lymphoid tissue and the thymic medulla) and circulating (blood, constituting 0.1% of the blood leukocytes, and the lymph) DCs.

The DCs in different locations have different forms and functions but all of them are professional APCs that constitutively express high levels of both class II MHC molecules and members of the co-stimulatory B7 family, namely the glycoproteins B7-1 and B7-2. This allows effective presentation of antigens together with class II MHC molecules to naive T_{H} cells and delivery of the co-stimulatory signal necessary for complete T cell activation, that leads to their proliferation and differentiation into effector T cells that carry out specialized functions. Because of these characteristics, DCs are considered as more potent APCs than macrophages and B cells, both of which need to be activated before they can function as APCs. Several other cell types, classified as nonprofessional APCs, can be induced to express class II MHC molecules and a co-stimulatory signal.

A considerable body of literature assigns a key role to dendritic cells (DCs) in promoting and modulating immune responses in general, and autoimmune responses in particular (Knight, et al., 2002; Link, et al., 2001). DCs are immune cells whose principal function is antigen presentation. They have an extraordinary capacity to stimulate naïve T cells, control the quality of the T cell response, and initiate primary immune responses (Mellman and Steinman, 2001). Their effects vary from conferring active autoimmunity to conferring immune tolerance, and they are capable of bringing about changes in T cell polarization (Dittel, et al., 1999; Turley, 2002; Xiao, et al., 2001).

The diverse activities of DCs in immune regulation are a function of the diversity of DC subsets and lineages, as well as the functional plasticity of DCs while still immature (Liu, 2001). The state of maturation of these cells, as well as their amount and the context in which they are activated, determines the nature of the resulting immune response. Three distinct stages of DC differentiation were recently described, and it was suggested that tolerance is conferred when the DCs are partially or semi-mature, whereas only fully mature DCs are immunogenic. The decisive signal, which induces a T cell-mediated immune response, seems to be the expression of CD86 (B7.2) and MHC class II (MHC-II) molecules concurrently with the release of proinflammatory cytokines, in particular interleukin (IL)-12, IL-6, and TNF-α, from the DCs (Lutz and Schuler, 2002).

Since DCs possess the unique ability to prime both naive helper and cytotoxic T cells, much interest has been fostered in their possible use in immune response modulation of infectious diseases, cancer, and autoimmune diseases. Dendritic cell-based vaccines have been proposed for immunotherapy of cancer and of bacterial, viral and other pathogen infections.

Citation or identification of any reference in any section of this application shall not be construed as an admission that such reference is available as prior art to the present invention.

### SUMMARY OF THE INVENTION

It has now been found, in accordance with the present invention, that local injection of dendritic cells pulsed with a peptide derived from the myelin basic protein sequence (MBP 87-99) or an analog of said peptide in which amino acid 91 was replaced by alanine (MBP-A91), resulted in dramatic recovery of rats after spinal cord contusion.

The present invention thus provides, in one aspect, a pharmaceutical composition comprising antigen-presenting cells (APCs) and a pharmaceutically acceptable carrier, wherein said APCs have been pulsed with an agent selected from the group consisting of:

(a) a nervous system (NS)-specific antigen or an analog thereof;

(b) a peptide derived from an NS-specific antigen or from an analog thereof, or an analog or derivative of said peptide;

(c) a copolymer selected from the group consisting of Copolymer 1, a Copolymer 1-related peptide, a Copolymer-1-related polypeptide and poly-Glu⁵⁰Tyr⁵⁰; and

(d) a non-self antigen.

The APCs according to the invention include, but are not limited to, human monocytes, macrophages, B cells and, more preferably, dendritic cells.

The pharmaceutical compositions according to the invention are useful for neuroprotection, namely, for preventing or inhibiting neuronal degeneration or for promoting nerve regeneration in the CNS or PNS, particularly for treating an injury, disorder or disease of the CNS including those that result in or is accompanied by axonal damage.

In another aspect, the present invention provides a method for neuroprotection, namely, for preventing or inhibiting neuronal degeneration in the CNS or PNS, which comprises administering to an individual in need thereof, an effective amount of APCs that have been pulsed with an agent selected from the group consisting of the agents (a) to (d) above.

In one particular embodiment, the method for neuroprotection is directed to preventing or inhibiting neuronal degeneration in any CNS injury, disorder or disease, including those that result in or is accompanied by axonal damage.

In a further aspect, the present invention provides a method for promoting nerve regeneration in the CNS or PNS, which comprises administering to an individual in need thereof, an effective amount of APCs that have been pulsed with an agent selected from the group consisting of the agents (a) to (d) above.

### BRIEF DESCRIPTION OF THE FIGURES

FIGS. 1A-1C show that the dendritic cells used in the experiments herein are mature. (FIG. 1A) FACS analysis of rat bone marrow-derived DCs on the first day of culture (d 0) and after culturing for 7 days in the presence of recombinant murine (rm)GM-CSF and rmIL-4. Only a few cells (1.6%) express both B7.2 (CD86) and MHC class II (OX6) molecules on d 0. Seven days later, most of the cells (94%) express large surface amounts of both CD86 and MHC class II molecules. (FIG. 1B) FACS analysis of rat bone marrow-derived DCs after culturing for 7 days in the presence of rmGM-CSF and rmIL4. The dashed line shows staining with control IgG antibodies and the light and dark lines show staining with markers of macrophages (ED-1) and B cells (CD45RA), respectively. As shown in the histogram, the cells do not express these markers. (FIG. 1C) RT-PCR of DCs cultured for 7 days before (left) and after (right) 2 h of pulsing with MBP-A91. Cultured DCs express IL-6, TNF-α and IL-12, which are markers of mature DCs, both before and after pulsing with MBP-A91. The values shown are from one of two experiments with similar results.

FIGS. 2A-2C show the effect of local injection of bone marrow-derived dendritic cells pulsed with myelin basic peptide MBP 87-99 or with MBP-A91 into rats with spinal cord contusion. (FIG. 2A) Injection of DCs pulsed with MBP 87-99 (circles) into the injury site, immediately after severe spinal cord contusion (NYU impactor, 10 g weight drop from a height of 50 mm), confers significant neuroprotection in male SPD rats (n=6 in each group) compared to PBS-injected controls. (FIGS. 2B, 2C) Injection of DCs pulsed with MBP-A91 into the site of the lesion (n=7) results in significantly improved locomotor performance. The neuroprotective effect of this treatment was statistically significant (*P≦0.05, **P ≦0.01, two-tailed Student's t-test; two-factor repeated measures ANOVA, P≦0.01, df=1, F-test=9.658). Note that the scores recorded in FIG. 2C are those of individual rats at the last time point examined.

FIGS. 3A-3B show lack of beneficial effect of local injection of bone marrow derived DCs unpulsed or pulsed with an irrelevant peptide on spinally contused rats. (FIG. 3A) Immediately after spinal cord contusion of male SPD rats, unpulsed DCs derived from bone marrow were locally injected into the site of injury (n=12). Control rats were similarly injected with PBS (n=10). The bars represent the mean BBB score of each group at the time when scores have reached a plateau (maximum values). Unpulsed DCs had no effect on recovery when compared to treatment with the vehicle PBS. Results are a pool of two experiments in which average control scores were identical. Four additional experiments showed the same pattern. (FIG. 3B) Immediately after spinal cord contusion the animals received a local injection of either PBS or bone marrow-derived DCs pulsed with ovalbumin (6 male SPD rats in each group). The bars represent the mean BBB score of each group at the time when scores have reached a plateau (maximal values). There is no significant difference in recovery between the two groups.

FIGS. 4A-4C show limited cavity formation after local implantation of dendritic cells pulsed with MBP peptides. Cyst areas in cryosections of spinal cord of vehicle-treated rats (4A) and of rats treated with DCs pulsed with MBP peptide (4B) (n=4 per group). Cyst areas were measured (Image-Pro Plus) in three slices taken from three planes in four spinal cords in each group (4C). Cyst areas differed significantly in the different groups (P<0.01, two-tailed Student's t-test), suggesting that treatment with DCs pulsed with MBP peptide significantly reduces the amount of syringomyelia (central cavitation of the spinal cord).

FIGS. 5A-5C show improved recovery as a result of local treatment with bone marrow-derived dendritic cells pulsed with altered myelin peptide after spinal injury in female Lewis rats. Immediately after severe spinal cord contusion, six female Lewis rats were locally injected with 5×10⁵ MBP-A91-pulsed DCs and five matched controls were injected with PBS. (5A) Injection of MBP-A91-pulsed DCs led to a significant improvement in locomotor performance measured by the BBB rating scale (*P≦0.05, **P≦0.01, two-tailed Student's t-test; two-factor repeated measures ANOVA, P≦0.01, df=1, F-test=8.701). (5B, 5C) Photomicrographs of Luxol fast blue-stained spinal cord sections from female Lewis rats treated with MBP-A91-pulsed DC cells (5B) or PBS (5C), with BBB scores of 8 or 5 respectively, 6 months after injury and treatment. These sections are representative of spinal cord sections from two DC-treated and two control rats that were analyzed histologically by Luxol fast blue staining. Note the significantly better preservation of neural tissue and the significantly smaller cysts in the treated rat.

FIG. 6 shows lack of neuroprotective activity by dendritic cells pulsed with altered myelin peptide MBP-A91in rats deprived of T cells. Three months after undergoing neonatal thymectomy, male Sprague-Dawley (SPD) rats (n=5 in each group) were subjected to spinal cord contusion and were then injected locally with 5×10⁵ MBP-A91-pulsed DCs or with PBS. In the absence of normal T cell fimction, the DCs had no significant effect on recovery. The results shown are representative of three experiments in thymectomized male and female SPD.

FIGS. 7A-7C show that intravenous (i.v.) administration of MBP-A91-pulsed dendritic cells promotes functional recovery after spinal cord injury. Sixteen SPD male rats were subjected to severe contusive SCI and were injected i.v. with 1×10⁶ MBP-A91-pulsed DCs or with PBS. (7A) Ten days later, three rats from each group were euthanized, their spleens were removed, and T-cell proliferation was assayed. Splenocytes from rats injected with DCs pulsed with MBP-A91, but not from PBS-injected rats, displayed a significantly stronger T cell response to MBP peptides (MBP 81-99, MBP 68-82, and A91) than to MOG 35-55, suggesting that injection of MBP-A91-pulsed DCs induces a T cell response directed to related myelin antigens. (7B) Intravenous injection of DCs pulsed with MBP-A91 resulted in significantly better functional recovery than that obtained after i.v. injection of PBS (n=5 in each group, two-factor repeated measures ANOVA, P≦0.003, df=1, F-test=18.43). (7C) Scores of individual rats at the last time point examined.

FIG. 8 shows that subcutaneous (s.c.) administration of MBP-A91-pulsed dendritic cells promotes functional recovery from spinal cord injury. Immediately after severe contusive SCI, female SPD rats (n=3 and 4) were injected s.c. with 2×10⁶ MBP-A91-pulsed DCs, or with PBS. The DC-MBP-A91-treated rats displayed significantly better locomotor performance than the control (*P≦0.05, **P≦0.01, two-tailed Student's t-test; two-factor repeated measures ANOVA, P≦0.01, df=1, F-test=12.353).

FIGS. 9A-9D show that there is a therapeutic window of 12 days for injection of dendritic cells pulsed with myelin peptides. A delay of 12 days in injecting rats paralyzed as a result of severe contusive SCI significantly improves functional recovery. (9A) Twenty SPD males underwent severe contusion, and 12 days later 11 rats with low locomotor scores were randomly assigned to two groups. Six rats received injections of 5×10⁵ MBP-A91-pulsed DCs into the injury site, while the other five were transplanted with 5×10⁵ DCs that were incubated in medium without MBP-A91. (9B) At all time points measured, from 29 days (17 days after treatment) to 160 days after the injury, the locomotor performance in the rats treated with MBP-A91-pulsed DCs was significantly better than in control rats (*P≦0.05, **P ≦0.01, two-tailed Student's t-test). The overall effect of the injection with DCs pulsed with myelin peptide was significant compared to the control (two-factor repeated measures ANOVA, P≧0.05, df=1, F-test=6.206). (9B) Scores of individual rats at the last time point examined. (9C, 9D) Using a similar experimental protocol, 5×10⁵ MBP-A91-pulsed DCs (n=6) or 5×10⁵ DCs (n=5) were administered locally into the injury site of spinally injured male SPD rats 28 days after SCI. No significant difference in recovery of the two groups was observed.

FIGS. 10A-10B depict maps showing diffusion anisotropy of the contused spinal cords. (10A) Nine months after SCI, the spinal cords were excised, fixed, and placed in 5-mm NMR tubes. The figure presents representative maps of contused spinal cords of rats that were locally injected with MBP-A91-pulsed DCs and control rats. Slices from left to right correspond to rostral-to-caudal axial slices. Colors correspond to anisotropy values. The maps show preservation of longitudinally ordered tissue at the lesion sites of the treated rats. Note that the site of injury in control rats is much larger than in rats from the treated group. The center of the injury site (asterisk) was determined by the slice with the lowest anisotropy value. (10B) Spatial distribution of the SAI (sum of anisotropy) value across slices. The figure shows the results for one representative rat of 2 in each group. Locomotor scores were 8.5 for the treated rat and 1.0 for the control.

### DETAILED DESCRIPTION OF THE INVENTION

According to the present invention, DCs specifically pulsed with peptides of myelin basic protein (MBP) were injected into the site of spinal cord contusion in rats. The purpose of this injection was to stimulate a well-regulated adaptive immune response against antigens that are abundant at the injury site. We postulated that the use of DCs pulsed with an MBP peptide might thus provide a way to harness the immune system and exploit its functions for both protection and regeneration of the injured spinal tissue (Hauben, et al., 2000; Rapalino, et al., 1998). In an attempt to obtain a beneficial outcome while reducing the risk of accompanying autoimmune disease, we also pulsed DCs with an altered peptide ligand, a segment of MBP (amino acids 87-99) in which the amino acid lysine in position 91 is replaced by alanine. This modified peptide (MBP-A91) has been shown to cross-react with the original encephalitogenic peptide, activating weak self-reacting T cells and thereby inducing autoimmunity without the risk of inducing experimental autoimmune encephalomyelitis (EAE) (Gaur, et al., 1997). When used as a vaccine in rats with injured spinal cords it can evoke protective autoimmunity (Hauben, et al., 2001b). The results showed that DCs pulsed with MBP-derived peptide, if injected either locally or systemically after SCI, promote recovery of locomotor activity. Recovery was also manifested by tissue preservation, measured morphologically and anatomically by MRI.

In one aspect, the present invention provides a pharmaceutical composition comprising human APCs and a pharmaceutically acceptable carrier, wherein said APCs have been pulsed with an agent selected from the group consisting of:

(a) a nervous system (NS)-specific antigen or an analog thereof;

(b) a peptide derived from an NS-specific antigen or from an analog thereof, or an analog or derivative of said peptide;

(c) a copolymer selected from the group consisting of Copolymer 1, a Copolymer 1-related peptide, a Copolymer-1 related polypeptide and poly-Glu⁵⁰Tyr⁵⁰; and

(d) a non-self antigen.

As used herein, the term “APCs” is intended to comprise, without limitation, monocytes obtained from peripheral blood; macrophages obtained from any site, including any tissue or cavity; macrophages derived by culturing macrophage precursors obtained from bone marrow or blood; dendritic cells (DCs) obtained from any site, including spleen, lymph node, skin and lymphatic fluid; DCs derived from culturing DC precursors obtained from bone marrow or blood; and B cells obtained from bone marrow or blood.

Human APCs can be obtained from the circulation or from any tissue in which they reside. Peripheral blood is an easily accessible ready source of monocytes, macrophages, DCs and B cells and is used as a source according to a preferred embodiment of the invention. APCs from other sources are well known in the art and include, without limitation, macrophages obtained from serosal cavities such as the peritoneal or pleural cavity, alveolar macrophages, and macrophages associated with other tissues, (e.g. liver, spleen, thymus) where they may be known by various terms such as Kupffer cells (in the liver) and microglial cells (in the CNS). APCs further include B lymphocytes and, more preferably, DCs.

In one embodiment, the APCs are human macrophages or monocytes, that can be prepared from blood as described in PCT/IL02/00930 of the present applicants, hereby incorporated by reference as is fully disclosed herein. The monocytes and macrophages may optionally be first stimulated by culturing the cells together with a tissue such as dermis, skin or nerve segment as described in the hereinbefore mentioned U.S. Pat. Nos. 5,800,812, 6,117,424 and 6,267,955.

In one preferred embodiment, the APCs are human DCs that can be obtained from any tissue where they reside including non-lymphoid tissues such as the epidermis of the skin (Langerhans cells) and lymphoid tissues such as the spleen, bone marrow, lymph nodes and thymus as well as the circulatory system including blood (blood DCs) and lymph (veiled cells). Human peripheral blood is an easily accessible ready source of human DCs and is used as a source according to a preferred embodiment of the invention. Cord blood is another source of human DCs and, if so desired, cord blood can be used as a source of DCs which can be cryopreserved for later use, if needed.

Especially preferred according to the invention is the use of autologous DCs purified from the peripheral blood of a subject to whom the therapeutic preparation is intended to be administered.

Because APCs, particularly DCs, occur in low numbers in any tissue in which they reside, including human peripheral blood, the APCs must be enriched or isolated for use. Enrichment techniques are well known to those skilled in the art and include, without limitation, elutriation; repetitive density gradient separation techniques such as centrifugation through material of suitable density, such as a Percoll gradient; positive selection, negative selection and combinations thereof; selective adhesion on suitable surfaces followed by removal at reduced temperature or at reduced concentrations of divalent cations, mechanical removal, or removal in the presence of lidocaine.

In one preferred embodiment, the APCs are obtained from peripheral blood by fractionation on Ficol and Percoll gradient and the monocyte-enriched fraction recovered from the Percoll interface is washed, resuspended in a suitable medium and cultured in Teflon bags at 37° C.

Once the DC's are obtained, they are cultured in appropriate culture medium to expand the cell population and/or maintain the DC's in a state for optimal antigen uptake, processing and presentation.

In humans, DCs comprise 3 subsets: Langerhans cells (LCs), localized in the basal and suprabasal layers of the epidermis, and interstitial or dermal DCs, present in the dermis and most organs, both in the myeloid lineage, and lymphoid DCs which are CD4⁺, CD11c−, CD13−, CD33−, and CD123⁺, and are present in blood and lymphoid organs.

The DCs according to the invention are preferably autologous DCs from the lymphoid subset. They can be isolated by standard techniques for isolating DCs from blood, bone marrow and lymphoid tissue. Preferably, at least 50%, more preferably at least 70%, still more preferably at least 80%, and yet more preferably at least 90% of the cells are DCs. Especially preferred is a substantially purified preparation of DCs e.g. a preparation in which at least 95% of the cells are DCs.

Generation of large numbers of highly pure DCs is possible by in vitro DC. culture systems. DCs can be cultured from CD34⁺ hematopoietic progenitors present in the bone marrow or peripheral blood (Caux et al., 1997, 1996) and also from three blood precursors differentiated from CD34⁺ progenitors: CD14⁺ monocytes (Sallusto et al., 1994), CD11c⁺ precursors, and CD11c− precursors (Geijtenbeek et al., 2000).

According to the invention, the DCs isolated from blood may be cultured without exogenous cytokines as described by Ho et al., 2002, or the DCs are cultured in a medium containing at least one stimulatory biologically active agent such as, but not limited to, transforming growth factor-beta (TGF-β), β-interferon (IFN-β), IFN-γ, tumor necrosis factor-α (TNF-α), interleukin 2 (IL-2), IL-3, IL-4, IL-6, IL-10, monocyte chemotactic and activating factor (MCAF), granulocyte colony-stimulating factor (G-CSF), macrophage colony-stimulating factor (M-CSF), granulocyte-macrophage colony-stimulating factor (GM-CSF), colony stimulating factor 1 (CSF-1), neurotrophic factor 3 (NT-3), nerve growth factor (NGF), brain-derived neurotrophic factor (BDNF), lipid A, the tripeptide fMet-Leu-Phe (fMLP), muramyl dipeptide (MDP), the calcium ionophore A23187, vitamin D3-binding protein, the T-cell analogue CD40 ligand (CD40L), as well as bacterial products such as lipopolysaccharide (LPS).

In one embodiment, functional DCs from blood or bone marrow CD34⁺ cells may be generated by a two-step culture combined with calcium ionophore treatment wherein in the first step the CD34⁺ hematopoietic progenitor cells are cultured in a medium in the presence of SCF, IL-3, IL-6 and G-CSF for about 10 days followed by culture for induction of DC in the presence of GM-CSF, IL-4 and TNF-α for 7-11 days and treatment with the calcium ionophore agent A23187 (Liu et al., 2002). The expression of co-stimulatory molecules (CD86, CD80) is up-regulated by the further treatment with the ionophore agent.

In another embodiment, DCs are cultured in a medium containing GM-CSF and IL-13 or, more preferably, in the presence of GM-CSF and/or IL-4. According to the invention, large amounts of DCs were obtained after 7 days in culture in the presence of GM-CSF and IL-4. In one preferred embodiment, for maintenance of the proper state of “maturity” of DCs in in vitro culture, the cells are cultured in the presence of GM-CSF, IL-4 or both, preferably in a combination of about 500 units/ml of each.

DCs can be cultured in any suitable cell culture device such as plastic tissue culture flasks or, more advantageously, in hydrophobic culture bags shown to be more suitable for the preparation of clinical DC vaccines, as DC can be generated, antigen-loaded, and matured in a close system (Guyre et al., 2002).

During maturation DCs undergo major changes in phenotype and function. There is a loss of endocytic and phagocytic receptors, whereas there are high levels of surface expression of MHC class II molecules, up-regulation of co-stimulatory molecules (CD80, CD86 and CD40) required for T-cell stimulation, and expression of CD83, a unique marker of matured DCs (Banchereau and Steinman, 1998). Several other molecules are also up-regulated, including adhesion molecules (ICAM-1 and VLA4). The processing of antigen (Ag) within late endosomes involves the degradation of foreign cells and infectious microorganisms into short peptides that are bound to membrane protein of MHC II. To maximize their Ag-presenting potential, mature DCs transiently increase the biosynthesis of MHC II molecules, and most strikingly, MHC molecules are massively exported to the cell membrane, where their half-life is prolonged as the rate of endocytosis is lowered (Pierre et al., 1997). The accumulation of high numbers of MHC II molecules on the cell membrane, together with increased expression of co-stimulatory molecules, allows for highly efficient Ag presentation to T lymphocytes. At the cell surface, these molecules remain stable for days and are available for recognition by CD4⁺ T cells.

In one embodiment of the present invention, the APCs, preferably DCs, preferably after activation as described above, are pulsed with a NS-specific, preferably CNS-specific, antigen or an analog thereof. The term “NS-specific antigen” as used herein refers to an antigen of the NS that specifically activates T cells such that following activation the activated T cells accumulate at a site of injury, disorder or disease in the NS of the patient. Examples of NS-specific antigens according to the invention include, but are not limited to, myelin basic protein (MBP), myelin oligodendrocyte glycoprotein (MOG), proteolipid protein (PLP), myelin-associated glycoprotein (MAG), S-100, β-amyloid, Thy-1, P0, myelin antigen P2, neurotransmitter receptors, the protein Nogo (Nogo-A, Nogo-B and Nogo-C) and the Nogo receptor (NgR). This definition also includes analogs of said NS-specific antigens.

In another embodiment, the APCs, preferably DCs, are pulsed with a peptide derived from an NS-specific antigen as defined above. As used herein, the term “peptide derived from an NS-specific antigen” means that the peptide has an amino acid sequence comprised within the sequence of the NS-specific antigen.

In a more preferred embodiment, the peptide is derived from the human MBP sequence (SEQ ID NO:1; Genbank accession number 307160), i.e. it has a sequence comprised within the MBP sequence. These MBP peptides include, but are not limited to, the peptides comprising the residues 75-95, 86-95, 82-98 and, preferably, the residues 87-99 of MBP. In one preferred embodiment, the MBP peptide is the peptide of SEQ ID NO:2 consisting of the amino acid residues 87-99 of human MBP (herein MBP 87-99,), of the sequence:

Val His Phe Phe Lys Asn Ile Val Thr Pro Arg Thr Pro

In another preferred embodiment of the present invention, the APCs, preferably DCs, are pulsed with an altered peptide, herein referred to as “altered peptide ligand” or “APL”, said altered peptide being an analog of a peptide derived from an NS-antigen in which critical amino acids in their TCR binding site, but not MHC binding site, are altered such that the altered peptide is non-encephalitogenic and still recognize the T-cell receptor.

In a more preferred embodiment, the modified CNS peptide according to the invention are analogs of the MBP peptide of SEQ ID NO:2 and include, but is not limited to, a peptide in which the Lys residue 91 of MBP 87-99 is replaced by Gly (MBP-G91; SEQ ID NO:3) or by Ala (MBP-A91; SEQ ID NO:4), or the Pro residue 96 is replaced by Ala (MBP-A96; SEQ ID NO:5), of the sequences:

Val His Phe Phe Gly Asn Ile Val Thr Pro Arg Thr Pro (SEQ ID NO:3)

Val His Phe Phe Ala Asn Ile Val Thr Pro Arg Thr Pro (SEQ ID NO:4)

Val His Phe Phe Lys Asn Ile Val Thr Ala Arg Thr Pro (SEQ ID NO:5)

Other analogs such as those derived from the residues 86 to 99 of human MBP by alteration of positions 91, 95 or 97 as disclosed in U.S. Pat. No. 5,948,764 for treatment of multiple sclerosis, are also encompassed by the present invention.

In a further embodiment, the APCs, preferably DCs, are pulsed with Cop 1 or a Cop 1-related peptide or polypeptide. Copolymer 1 or Cop 1 is a random copolymer composed of the four amino acids: tyrosine-glutamate-alanine-lysine, that cross-reacts functionally with MBP and is able to compete with MBP on the MHC class II in the antigen presentation. Cop 1, in the form of its acetate salt known under the generic name Glatiramer acetate, has been approved in several countries for the treatment of multiple sclerosis under the trade name COPAXONE® (Teva Pharmaceuticals Ltd., Petah Tikva, Israel). Cop-1 related polypeptides can be prepared as described in U.S. patent application Ser. Nos. 09/756,301 and 09/765,644, both dated 22 Jan., 2001, hereby incorporated by reference in their entirety as if fully disclosed herein, and Cop-1 related peptides are disclosed in WO/005249, the entire contents of which are hereby incorporated herein by reference.

In yet another embodiment, the APCs, preferably DCs, are pulsed with poly-Glu⁵⁰Tyr⁵⁰, formerly called polyGT and also designated pEY, described in the hereinbefore mentioned WO 03/022140 of the present applicants.

In yet a further embodiment, the APCs, preferably DCs, are pulsed with a non-self antigen such as, but not limited to, ovalbumin and tetanus toxin. DCs pulsed with the non-self antigen are then implanted locally in an individual in need that has been previously immunized with said non-self antigen. Usually, the immunization with the non-self antigen will be performed soon after the injury occurrence and the implantation of the pulsed DCs at the lesion site will be made 6-14 days thereafter. In this way, the T cells specific to the non-self antigen will get to the site of the lesion among T cells of other specificities but only said specific T cells will be activated by the antigen exposed on the APCs and will display their neuroprotective effect. In this respect, it is interesting to note that T cells specific to the non-self antigen ovalbumin were shown by the present inventors to accumulate at the site of injury but had no neuroprotective effect (Hirschberg et al., 1998). However, according to the present invention, when ovalbumin is presented by the APCs implanted at the site of injury, the ovalbumin-specific T cells generated by previous immunization with ovalbumin, will accumulate at the site of injury, will become activated and will exert their neuroprotective effect. When the non-self antigen is tetanus toxoid, usually the immunization soon after the injury will not be necessary because most of the individuals in a population have been immunized once with tetanus toxoid.

According to the invention, after culture, DCs can be used immediately or they can be preserved by freezing. For further use, DCs are collected, treated with a cryopreservative, for example a solution containing 10% dimethyl sulfoxide and 2% human albumin, and cryopreserved in bags either by putting the bags directly in a mechanical freezer at −80° C. or using a classical liquid nitrogen controlled-rate freezer at −1° C./min, and stored. When needed, the frozen preparation is thawed and administered to the patient. The DCs can also be frozen before pulsing with the antigen and stored, and are then pulsed with the antigen and administered to the patient. Studies have shown that the immunophenotype of DCs as well as the T cell-stimulating capacity were not modified by the freezing and thawing of DCs (Garderet et al., 2001).

For the preparation of the pharmaceutical compositions of the invention, the antigen-pulsed cells are suspended in a sterile pharmaceutically acceptable carrier. In a preferred embodiment, the pharmaceutically acceptable carrier is PBS, a culture medium, or any pharmaceutically acceptable fluid in which the cells are suspended. However, alternative pharmaceutically acceptable carriers will readily be apparent to those skilled in the art.

The pharmaceutical compositions of the invention are useful for preventing or inhibiting neuronal degeneration, or for promoting nerve regeneration, in the central nervous system (CNS) or peripheral nervous system (PNS) and, in particular, for preventing or inhibiting neuronal degeneration caused by any injury, disorder or disease of the CNS or PNS including those that result in or is accompanied by axonal damage.

The injury, disorder or disease may be situated in any portion of the PNS or CNS, including the brain, spinal cord, or optic nerve. One example of such injury, disorder or disease is trauma, including spinal cord injury, blunt trauma, brain coup or contrecoup injury, penetrating trauma, and trauma sustained during a neurosurgical operation or other procedure. Another example of such injury, disorder or disease is stroke, including hemorrhagic stroke and ischemic stroke. Yet another example of such injury, disorder or disease is a disease associated with the eye, e.g. glaucoma, age-related macular degeneration, optic neuropathy, or retinal degeneration. Still further examples of PNS or CNS injury, disorder or disease include diabetic neuropathy, senile dementia, Alzheimer's disease, Parkinson's disease, facial nerve (Bell's) palsy, Huntington's chorea, amyotrophic lateral sclerosis (ALS), vitamin deficiency, epilepsy, amnesia, anxiety, hyperalgesia, psychosis, seizures, oxidative stress, and opiate tolerance and dependence.

The compositions and methods of the present invention are useful for treating CNS injury, disorder or disease that may result in axonal damage whether or not the subject also suffers from other disease of the central or peripheral nervous system, such as a neurological disease of genetic, metabolic, toxic, nutritional, infective or autoimmune origin.

The present invention further provides a method for preventing or inhibiting neuronal degeneration, or for promoting nerve regeneration, in the CNS or PNS, which comprises administering to an individual in need thereof an effective amount of a pharmaceutical composition comprising the antigen-pulsed cells, preferably, antigen-pulsed dendritic cells, as described herein.

The NS-antigen-pulsed APCs, preferably DCs, can be administered to the patient locally or systemically, for example, intravenously, subcutaneously, intradermally, intratracheally or intranasally.

In a preferred embodiment, the APCs, preferably DCs, are administered immediately following CNS injury and are introduced at or near the site of CNS injury, by any neurosurgically suitable technique, for example with a glass micropipette or a syringe. However, the present invention encompasses administration of DCs also at any time, e.g. within a week, a fortnight, or even more, after the CNS sustains injury, disorder or disease.

The optimal dose of antigen-pulsed cells for use in humans may be deduced from the experiments in rats described herein, according to which the optimal dose for treatment of injury in the spinal cord by local administration was found to be 5×10⁵ DCs, 10⁶ DCs for i.v. administration, and 2×10⁶ DCs for s.c administration per rat spinal cord. As will be evident to those of skill in the art, the dose of cells can be scaled up or down in proportion to the number of nerve fibers affected at the lesion or site of injury being treated. For humans, the amount of cells and number of injections would have to be calculated according to the migration properties of the cells and the area of damaged fibers. For treatment of neurodegenerative diseases, the number of DCs to be injected should be calculated per area of damaged tissue.

In one embodiment of the present invention, in an attempt to obtain a beneficial outcome without the risk of accompanying autoimmune disease, DCs were used that were specifically pulsed with myelin antigens, namely the peptide MBP 87-99. Alternatively, DCs were used that were pulsed with the altered peptide ligand designated MBP-A91, that has been shown to cross-react with the original encephalitogenic peptide without inducing experimental autoimmune encephalomyelitis (EAE), and when applied to rats with injured spinal cords was able to induce a protective autoimmunity (Hauben et al., 2001b).

The results described herein showed a dramatic recovery from spinal cord contusive injury in rats treated by local injection of bone-marrow-derived DCs pulsed in vitro with the myelin-derived encephalitogenic peptide MBP 87-99 or the non-encephalitogenic MBP-A91. The improved functional recovery was manifested by an increase in functional activity measured by locomotion in an open field, and by enhanced survival of neural tissue measured morphologically and immuno-cytochemically. The morphological analysis further demonstrated reduction in cavity formation and increased sprouting.

Recognizing that recovery from spinal cord injury can be facilitated by boosting a well-regulated local immune response, the present invention examined the effectiveness of local injection of efficient APCs, namely DCs, committed to myelin antigen, rather than injecting T-cells activated by antigen or systemic vaccination. The results herein showed a significantly improved recovery in spinally injured rats injected with DCs pulsed with selected peptides compared to vehicle-injected controls.

Injury-induced self-destructive processes cause significant functional loss after incomplete spinal cord injury (SCI). Cellular elements of both the innate (macrophage) and the adaptive (T cell) immune response can, if properly activated and controlled, promote post-traumatic regrowth and protection after SCI. Dendritic cells (DCs) trigger activation of effector and regulatory T cells, providing a link between the functions of the innate and the adaptive immune systems. They also initiate and control the body's response to pathogenic agents and regulate immune responses to both foreign and self-antigens. It is shown herein in the present application that post-injury injection of bone marrow-derived DCs pulsed with encephalitogenic or non-encephalitogenic peptides derived from MBP, when administered (either systemically or by local injection into the lesion site) up to 12 days after the injury, led to significant and pronounced recovery from severe incomplete SCI. No significant protection was seen in DC recipients deprived of mature T cells. Flow cytometry, RT-PCR, and proliferation assays indicated that the DCs prepared and used here were mature and immunogenic. Taken together, the results indicate that the DC-mediated neuroprotection was achieved through the induction of a systemic T cell-dependent immune response. Better preservation of neural tissue and diminished formation of cysts and scar tissue accompanied the improved functional recovery in DC-treated rats. The use of antigen-specific DCs may represent an effective way to obtain, via transient induction of an autoimmune response, the maximal benefit of immune-mediated repair and maintenance, as well as protection against self-destructive compounds.

Without wishing to limit the scope of the present invention, the following examples are provided to illustrate preferred aspects of the invention

### EXAMPLES

Materials and Methods

(a) Animals

Inbred adult Lewis or Sprague-Dawley (SPD) rats (10-12 weeks old, 200-250 g) were supplied by the Animal Breeding Center of The Weizmann Institute of Science (Rehovot, Israel). Rats were housed in a light- and temperature-controlled room and were matched for age in each experiment. All animals were handled according to the guidelines of the National Institutes of Health and The Weizmann Institute of Science for the management of laboratory animals.

(b) Antigens

Modified (non-encephalitogenic) MBP peptides were derived from an encephalitogenic peptide, amino acids 87-99 of MBP, by replacing the lysine residue 91 with alanine (A91, synthesized at the Weizmann Institute of Science, Rehovot, Israel). All peptides used in the experiments had a purity of>95% as confirmed by reverse-phase HPLC (RP-HPLC). Ovalbumin (OVA) was purchased from Sigma, Israel.

(c) RT-PCR (Reverse Transcription-Polymerase Chain Reaction)

Total RNA was extracted using TRI Reagent®. For the first-strand cDNA synthesis reaction, RNA was incubated at 65° C. for 5 min, chilled on ice, and then reverse-transcribed in the presence of oligo-dT primer, 50 mM Tris-HCl pH 8.3, 75 mM KCl, 3 mM MgCl₂, 20 mM DTT, 0.5 mM dNTP mixture, and 200 U of SuperScript II Rnase Reverse Transcriptase (Life Technologies, Rockville, Md.) at 42° C. for 1 h. The generated cDNA was amplified with 0.6 U of DyNAzyme II DNA polymerase (Finnzymes Oy, Rihitontuntie, Finland) in the presence of 50-70 pmol of primers, 0.1 mM dNTP mixture, 10 mM Tris-HCl pH 8.8, 1.5 mM MgCl₂, 50 mM KCl, and 0.1% Triton X-100. The cycling conditions were 30 sec at 94° C. for denaturation, 1 min at 60° C. for annealing, 2 min at 72° C. for extension, and 7 min at 72° C. after the last cycle. cDNA samples were amplified by 24 cycles for L-19, 34 cycles for IL-6, 33 cycles for TNF-α, and 34 cycles for IL-12 p-40. PCR products were visualized after electrophoresis on 1.5% agarose gels by staining with ethidium bromide. The following primers were used:

| | | |
|---|---|---|
| L-19: | 5′CTGAAGGTCAAAGGGAATGTG | (SEQ ID NO: 6) |
| and | | |
| | 5′GGACAGAGTCTTGATGATCTC; | (SEQ ID NO: 7) |
| IL-6: | 5′ACTGCCTTCCCTACTTCAC | (SEQ ID NO: 8) |
| and | | |
| | 5′GTATTGCTCTGAATGACTCTG; | (SEQ ID NO: 9) |
| TNFα: | 5′AGGAGGCGCTCCCCAAAAAGATGGG | (SEQ ID NO: 10) |
| and | | |
| | 5′GTACATGGGCTCATACCAGTTG; | (SEQ ID NO: 11) |
| IL-12: | 5′AGATGACATCACCTGGACCT | (SEQ ID NO: 12) |
| and | | |
| | 5′CTTTGGTTCAGTGTGACCTTC. | (SEQ ID NO: 13) |

(d) Preparation of Rat Dendritic Cells

DCs were generated from bone marrow by a previously described method (Lutz, et al., 1999; Talmor, et al., 1998), with some modifications. Femurs and tibias were removed from euthenized mature male SPD rats (7-10 weeks old), stripped of muscle and connective tissue, placed in 70% ethanol for 3 min for disinfection, and then washed with phosphate-buffered saline (PBS). Both ends of the bones were cut with scissors and the marrow was flushed out with calcium-free and magnesium-free PBS using a syringe with a 23-gauge needle. Cell aggregates were broken down by vigorous pipetting. Red blood cells were lysed with ACK buffer. Bone marrow cells were counted, and plated at 2-5×10⁶ cells per ml in a 250-ml flask (total 15 ml). The cells were grown in Dulbecco's modified Eagle's medium (DMEM) supplemented with penicillin and streptomycin (100 μg/ml), L-glutamine (2 mM), β-mercaptoethanol (50 μM), pyruvate (1 mM), non-essential amino acids (1:100), and 10% heat-inactivated and filtered fetal calf serum (referred to hereafter as DC medium). The cytokines recombinant murine granulocyte macrophage colony-stimulating factor (rmGM-CSF, PeproTech, Rocky Hill, N.J.) and recombinant murine interleukin 4 (rmIL-4, PeproTech), both at 20 ng/ml, were added on day 0. On days 2 and 4 the culture medium was replaced with DC medium supplemented with cytokines and the floating cells were discarded. On day 7 cells were collected and centrifuged, and the pellet was resuspended in fresh DC medium (without added cytokines; 2×10⁶ cells per ml) containing the specific antigen (20 μg/ml). The cells were pulsed (i.e., incubated for 2 h with the antigen), washed with fresh DC medium, and kept on ice until injected. Just before injection the cells were centrifuged and resuspended in PBS (5×10⁵ cells in 5 μl PBS for local injection; 1×10⁶ cells in 1 ml PBS for intravenous (i.v.) injection; 2×10⁶ cells in 1 ml PBS for subcutaneous (s.c.) injection). For local injection the cells were loaded into a Hamilton syringe and injected into the spinal cord at the site of injury. For s.c. injection, cells were injected in the neck area at two injection sites (0.5 ml each). For i.v. injection, cells were injected into the tail vein.

(e) Spinal cord injury

Rats were anesthetized by intramuscular injection of Rompun (xylazine, 10 mg/kg; Vitamed, Israel) and Vetalar (ketamine, 50 mg/kg; Fort Dodge Laboratories, Fort Dodge, Iowa) and their spinal cords were exposed by laminectomy at the level of T8. One hour after induction of anesthesia, a 10-g rod was dropped onto the laminectomized cord from a height of 50 mm (defmed as a “severe” injury), using the NYU impactor, a device shown to inflict a well-calibrated contusive injury of the spinal cord (Basso, et al., 1996; Hauben, et al., 2000; Hauben, et al., 2000; Young, 1996).

(f) Experimental Protocolfor DC Administration

Bone marrow-derived cultured DCs were pulsed with MBP peptide 87-99, MBP-derived altered peptide A91, or with ovalbumin (20 μg/ml) for 2 h, washed with PBS, and adjusted to the appropriate number and volume just before injection. The spinal cords of SPD or Lewis rats were contused at the level of T9, using the NYU impactor, as described above in (e). Rats in the control group were injected with 5 μl of PBS or with non-pulsed DCs. Immediately after SCI, the treated groups were injected locally into the injury site, or subcutaneously at two adjacent sites in the neck area, or intravenously into the tail vein, with DCs in PBS at the concentrations recorded above in (d). Control rats were injected, locally or subcutaneously or intravenously, respectively, with the same volume of PBS as the treated rats.

To examine the effect of delayed treatment, rats were anesthetized 12 or 28 days after SCI, the laminectomized area was exposed, and the injured spinal cord was further exposed by careful separation of the healed tissue over the spinal cord. DCs or PBS were then injected into the spinal cord as described above.

(g) Assessment of Recovery from Spinal Cord Contusion.

Functional recovery was determined by locomotor hindlimb performance. This was scored using the open-field locomotor rating scale of Basso, Beattie, and Bresnahan (BBB), on a scale of 0 (complete paralysis) to 21 (normal mobility) (Basso, et al., 1996; Hauben, et al., 2001; Hauben, et al., 2000; Jakeman, et al., 2000; Ma, et al., 2001; Young, 1996). Blind scoring ensured that observers were not aware of the treatment received by each rat. Approximately once a week, we evaluated the locomotor activities of the trunk, tail, and hind limbs in an open field by placing the animal for 4 min in the center of a circular enclosure (90 cm diameter, 7 cm wall height) made of molded plastic with a smooth, non-slip floor. Prior to each evaluation the rat was examined carefully for perineal infection, wounds in the hind limbs, and tail and foot autophagia.

(h) Animal Care

In spinally injured rats, bladder expression was assisted manually twice a day (three times a day during the first 48 h after injury) until the end of the 2^{nd} week, by which time automatic voidance had been recovered. Rats were carefully monitored for evidence of urinary tract infection or any other sign of systemic disease. During the first week after contusion and in any case of hematuria after that period, they received a course of sulfamethoxazole (400 mg/ml) and trimethoprim (8 mg/ml) (Resprim, Teva Laboratories, Israel), administered orally with a tuberculin syringe (0.3 ml of solution per day). Daily inspections included examination of the laminectomy site for evidence of infection and assessment of the hind limbs for signs of autophagia or pressure.

(i) Histology

At the indicated time points, rats were perfused intracardially with 100 ml of cold 0.1 M PBS, pH 7.4, at 4° C., and then with 200 ml of 4% paraformaldehyde (prepared in 0.1 M PBS, pH 7.4, containing glucose 5%). Their spinal cords were removed, postfixed overnight in 10% phosphate-buffered formaldehyde, dehydrated overnight in ethanol, and embedded in a paraffin block. Serial sections (4 μm) from each block were stained with hematoxylin and eosin or Luxol fast blue.

(j) Proliferation Assay

Three rats from each group were euthanized 12 days after injury, and their spleens were excised and pressed through a fine wire mesh. After lysis of red blood cells with ACK lysing buffer (BioSource, USA), the splenocytes were washed in PBS and resuspended in proliferation medium containing DMEM supplemented with L-glutamine (2 mM), β-mercaptoethanol (5×10⁻⁵M), sodium pyruvate (1 mM), penicillin (100 IU/ml), streptomycin (100 μg/ml), non-essential amino acids, and autologous rat serum 1% (vol/vol). Splenocytes were cultured in quadruplicate in flat-bottomed microtiter wells in 100 μl of medium (3×10⁶ cells/ml) with concavalin A (Con A; 1.25 μg/ml) or MBP 81-99 (10 μg/ml) or MBP 68-82 (10 μg/ml) or MBP-A91 (10 μg/ml) or MOG 35-55 (10 μg/ml) or without antigen for 72 h at 37° C., 90% relative humidity and 7% CO₂. The proliferative response was determined by measuring the incorporation of ³[H]thymidine (1 μCi/well), which was added to each well for the last 16 h of the 72-h culture.

(k) Diffusion-Anisotropy Magnetic Resonance Imaging

Rats were euthanized and their spinal cords were excised and examined by magnetic resonance imaging (MRI). Diffusion anisotropy was measured in a Bruker DMX 400 widebore spectrometer, using a microscopy probe with a 5-mm Helmholz coil and actively shielded magnetic field gradients. The observer was blinded to the identities of the rats. Multislice echo imaging was performed with 9 axial slices, with the central slice positioned at the center of the spinal injury. Images were obtained with a TE of 31 ms, TR of 2000 ms, diffusion time 15 ms, diffusion gradient duration 3 ms, field of view 0.6 cm, matrix size 128×128 pixels, slice thickness 0.5 mm, and slice separation 1.18 mm. Left to right images represent axial sections from head to foot. Four diffusion gradient values (0, 28, 49, and 71 g/cm) were applied along the read direction (transverse diffusion) or along the slice direction (longitudinal diffusion). Using a linear fit by weighted minimal squares for each pixel, we obtained a transverse (T) and a longitudinal (L) apparent diffusion coefficient map, from which an anisotropy ratio matrix was derived. The accumulated anisotropy in each slice was integrated. For each rat, the lowest value of the slice anisotropy integral was defined as the lesion site.

(l) Measurement of Cyst Area

Prior to longitudinal sectioning of the spinal cord, each rat was perfused intracardially as described in (i). The spinal cords were removed, post-fixed overnight in 4% paraformaldehyde (prepared in 0.1 M PBS, pH 7.4, containing glucose 5%), rinsed briefly in PBS, and transferred to sucrose 30% for cryoprotection for at least 3 days. All procedures were carried out at 4° C. A 20-mm block of the spinal cord, with the injury site in the middle, was excised, embedded in Tissue-Tek (Miles, Ind.), and placed in liquid nitrogen. The frozen spinal cord blocks were longitudinally sectioned (20 μm thickness) on a cryostat, collected onto gelatin-coated slides, and dried at room temperature. The sections were treated for 1 min with a solution of 0.3% Sudan Black B (Merck, Darmstadt, Germany) in 70% ethanol. If overstained, the sections were dipped in fresh 70% ethanol until staining was satisfactory. The slides were stored in a dry box at 4° C. pending further analysis. 50 sections from each spinal cord (n=4 for each group) were inspected, out of which the 5^{th}, 25^{th}, and 45^{th} section, representing the bilateral and midsagittal areas of interest were selected for further quantitative analysis. The sizes of cysts were determined by a semi automated image analysis. The borders of the spinal cord sections were defined manually and the number of blank pixels (i.e. no tissue within) were thereby automatically measured (Image-Pro Plus program), yielding the accumulated size of cysts (each pixel is 1.8×1.8 μm²)

(m) FACS Analysis

Bone marrow-derived cultured DCs (5×10⁵) were stained with CD86-FITC (anti B7.2, mouse IgGlk, Pharmingen, San Diego, Calif., USA), OX6 (anti MHC-II mouse IgGlk, Pharmingen), ED-1 (Serotec, Oxford, U.K), CD45RA (Pharmingen), and their control antibodies. The cells were incubated in 100 μl of PBS containing 2% normal mouse serum and the diluted specific antibodies at 4° C. for 30 min. Cells were washed with 4 ml of PBS and re-suspended in 400 μl of 0.1% PFA solution. Samples were analyzed with a FACScan (Becton-Dickinson, Heidelberg, Germany). Staining for ED-1 was performed using the Leucoperm reagents (Serotec, Oxford, U.K.) according to manufacturer protocol.

(n) Statistical Analyses

Behavioral and morphological data were analyzed by two-tailed Student's t-tests. Since the open-field motor scores were measured at different times after the injury, they were also analyzed by two-factor repeated-measures ANOVA.

### Example 1

Characterization of the Bone Marrow Derived DC's

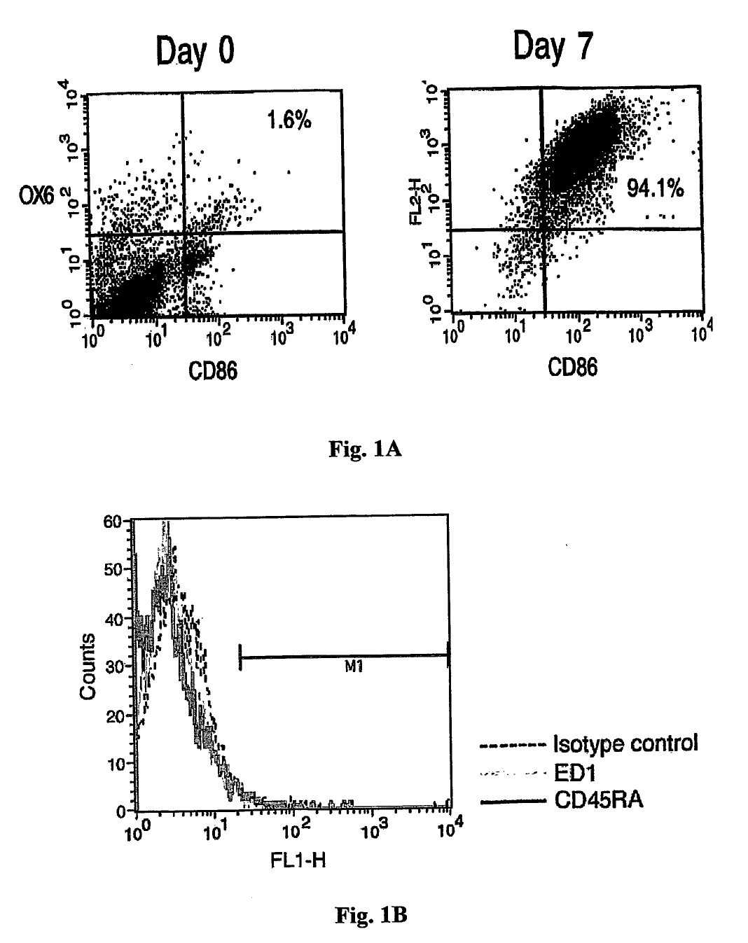

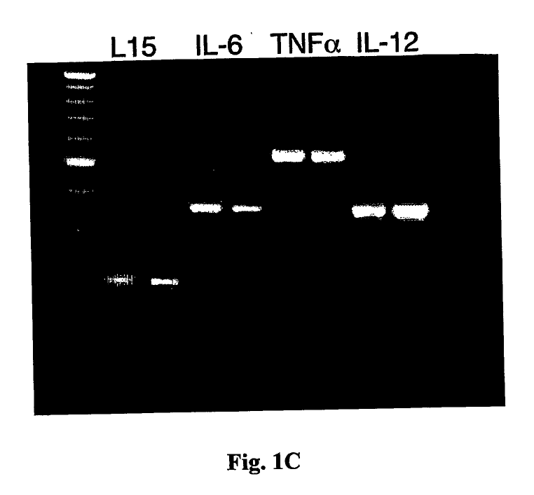

We first characterized the purity of the DC preparation as well as the maturity of the cells. Bone marrow-derived DCs were analyzed by flow cytometry for expression of the costimulatory B7.2 (CD86) and MHC-II molecules on their surface. As shown in FIG. 1A, most of the cells (94%) expressed B7.2 and MHC-II at the time of their harvesting for injection (day 7), whereas on the day that culture was initiated (day 0) these DC markers were expressed by only 1.6% of the cells.

Other cell types that may express high levels of B7.2 and MHC-II include macrophages and B cells. We therefore analyzed cultures on day 7 by Flow cytometry for the expression of ED-1, a marker for macrophages, and for CD45RA, a marker for B cells. The histogram in FIG. 1B shows that the cells were negative for both these markers.

We also examined the degree of DC maturation and whether it is affected by the exposure to the antigen. RNA was extracted from DCs before and after the cells were pulsed with MBP-A91, and subjected to RT-PCR to detect expression of the cytokines INFα, IL-12 and IL-6, all of which are known to be expressed by mature DCs but not by semi-mature or immature DCs (Lutz and Schuler, 2002). As shown in FIG. 1C, the MBP-A91-pulsed DCs expressed all three of these cytokines and were therefore characterized as mature. Non-pulsed DCs also expressed the same cytokines. It thus seems that the DCs used in the present example are mature, and that their maturity was not dependent on pulsing with the antigen.

### Example 2

Effect of Dendritic Cells Pulsed with MBP 87-99 or its Analog MBP-A91 on Rats Subjected to SCI: Local Implantation of Bone Marrow-Derived DCs Exposed to Myelin Peptide Promotes Functional Recovery from SCI

Male SPD rats were subjected to a severe contusive injury as described in Methods, section (e). Rats were treated immediately after the injury by local injection with bone marrow-derived DCs pulsed (by incubation for 2 h) with MBP peptide 87-99 or with the modified (and therefore no longer encephalitogenic) peptide MBP-A91, as described in Methods. Control groups were locally injected with the vehicle (PBS). Functional recovery was assessed by the BBB locomotor rating scale on a scale of 0-21 (Basso et al., 1996), where 0 denotes no mobility and 21 denotes full mobility Blind scoring ensured that the identity of the rats was masked.

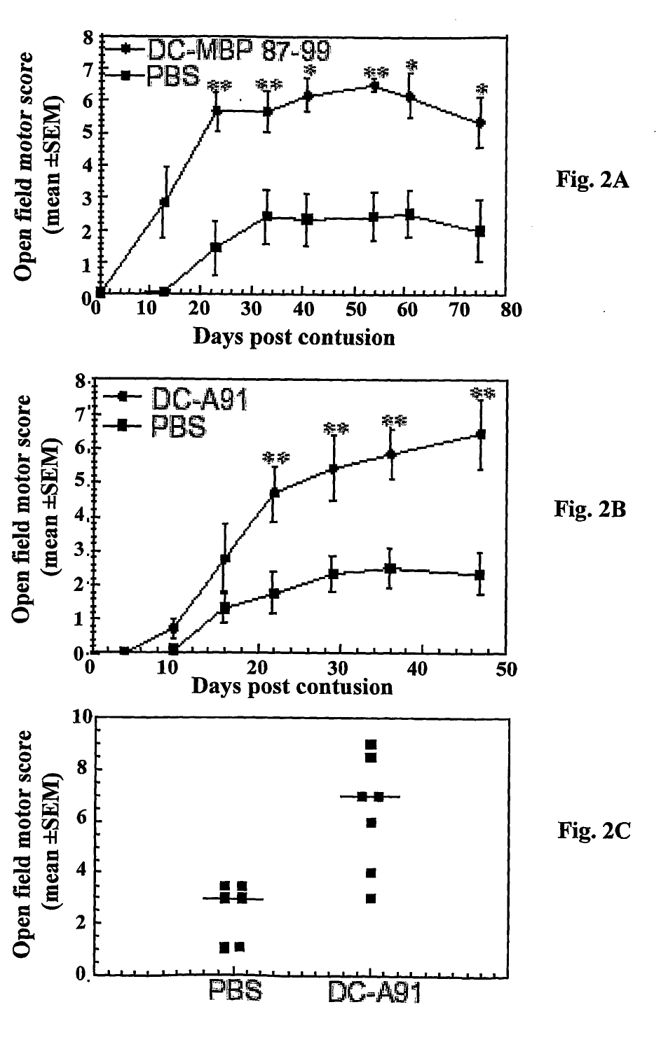

After severe contusion and local PBS injection, the rats showed extremely limited recovery from the initial shock (FIG. 2). However, injured rats treated with DCs pulsed with either MBP 87-99 (FIG. 2A) or MBP-A91 (FIG. 2B) showed a significantly improved recovery, which was detectable as early as 11 days after contusive SCI (FIGS. 2A, 2B). Recovery was evident in 70% to 80% of the treated rats, some of which attained locomotion scores as high as 9 (FIG. 2C), manifested by extensive movement of all three hindlimb joints (BBB score of 7), plantar placement of the paw (BBB=8), and weight support when stationary (BBB=9). The mean BBB score±SEM in the treated rats was 6.4±0.9 (FIGS. 2B,2C). In control rats, the highest BBB score obtained in this set of experiments was 4, manifested by slight movements of the hindlimb joints, and the mean score was 2.3±0.6.

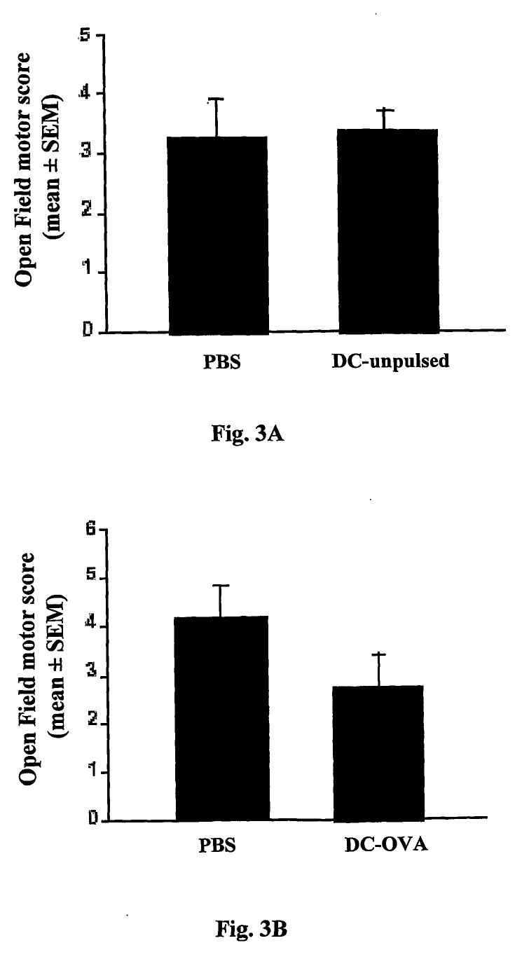

To examine whether antigenic specificity plays a role in the ability of the locally injected DCs to promote spinal cord recovery, we tested the effect of unpulsed DCs and DCs pulsed with an irrelevant antigen such as ovalbumin in two separate sets of experiments. The results revealed lack of a significant difference in the recovery of animals treated with unpulsed DCs relative to PBS-treated controls (FIG. 3A). Similar results were obtained in 4 additional independent experiments. DCs pulsed with ovalbumin also did not have any effect on recovery (FIG. 3B).

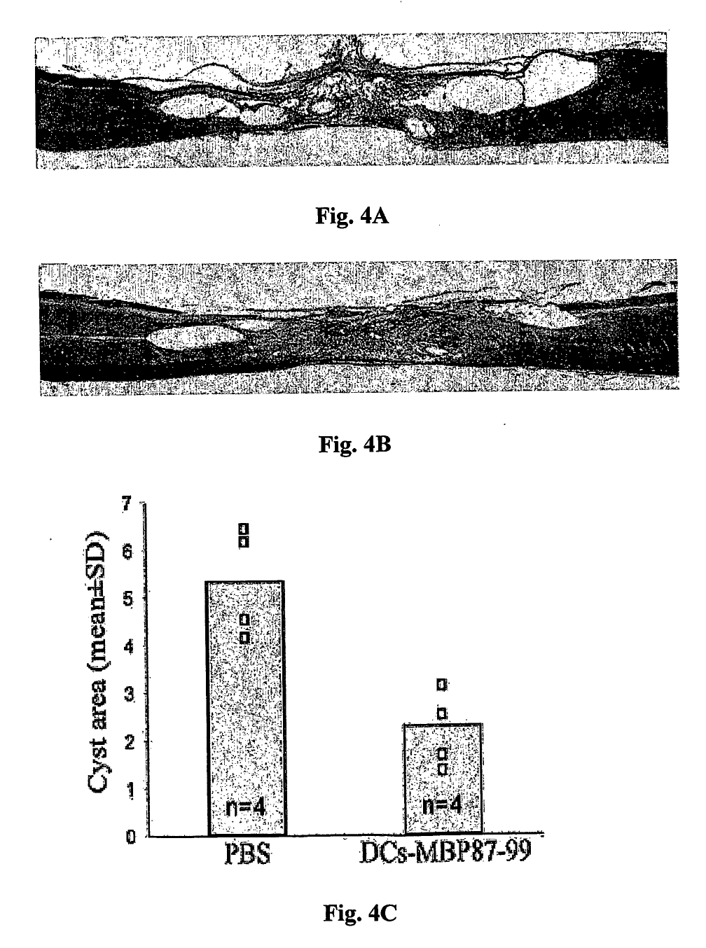

Histological analysis of rat spinal cords excised 3.5 months after severe spinal contusion and local injection of DCs pulsed with MBP peptide 87-99 or PBS injection revealed significantly better tissue preservation in the DC-treated rats (n=4) than in the controls (n=4), manifested by less cavitation and smaller sites of injury (FIG. 4A vs. 4B). Lesion sites in the treated rats were significantly smaller (by 2- to 3-fold) than in the controls. Approximately 50 slices from each spinal cord were inspected and all showed the same pattern: the cyst areas in the DC-treated spinal cords were smaller than in the untreated spinal cords. Quantification of cysts was taken from three different slices in each spinal cord representing three planes of sectioning, with the same planes represented in all spinal cords. The mean values of the three slices in the four different animals are shown in FIG. 4C. A comparison of the cyst areas revealed significant differences, suggesting that treatment with DCs pulsed with MBP peptide reduces the amount of syringomyelia (central cavitations of the spinal cord).

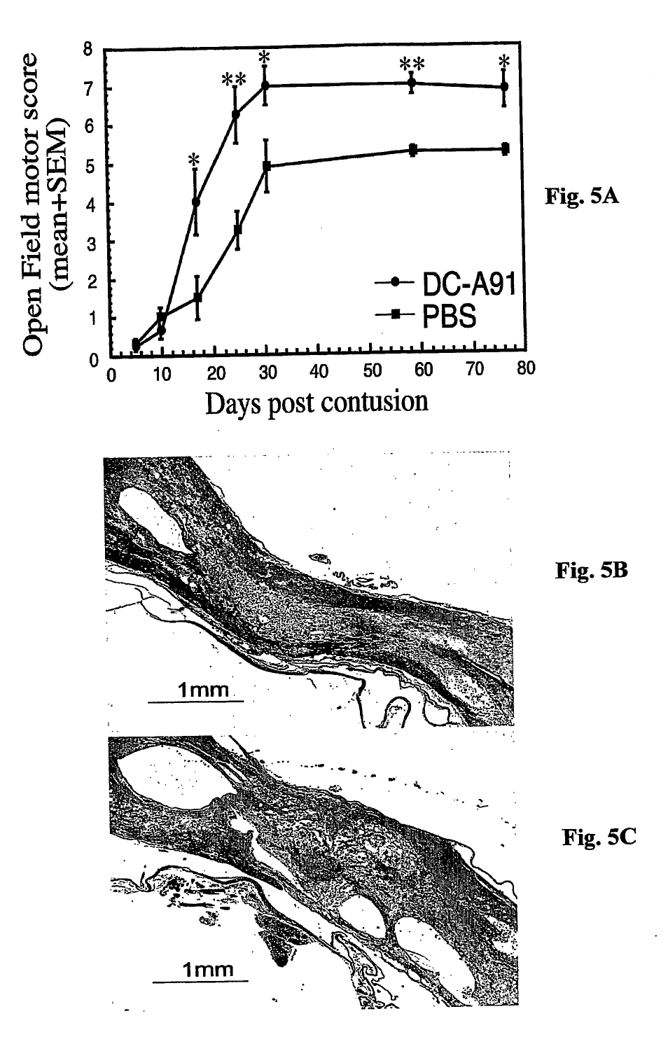

Differences in the spontaneous recovery from SCI have been reported between rat strains that are susceptible and rat strains that are resistant to the induction of autoimmune disease (Hauben, et al., 2001; Kipnis, et al., 2001), as well as between male and female mice or rats (Hauben, et al., 2002). It was therefore of interest to examine the effect of the treatment with DCs pulsed with MBP-derived peptide in female Lewis rats, an EAE-susceptible strain. FIG. 5 illustrates the significant effect of post-traumatic local injection of MBP-A91-pulsed DCs on the functional recovery of six spinally injured female Lewis rats compared to five PBS-injected female littermates (two-factor repeated measures ANOVA, P≦0.01, df=1, F-test=8.701). The highest BBB score of the PBS-injected controls was 5.2±0.2 (mean±SEM), whereas rats injected with MBP-A91-pulsed DCs reached a maximum mean score of 7.2±0.4 (P≦0.005, two-tailed Student's t test) (FIG. 5A). Six months after SCI, the spinal cords of two rats from each group were excised and processed for histological analysis. Approximately 20 sections (4 μm) from each spinal cord were inspected. Representative sections from each group are shown in FIGS. 5B and 5C. The spinal cords of rats treated with MBP-A91-pulsed DCs showed better preservation of neuronal tissue and less cavitation.

### Example 3

An Insight Into the Immunological Mechanism Underlying the DC-Induced Recovery from Spinal Cord Injury

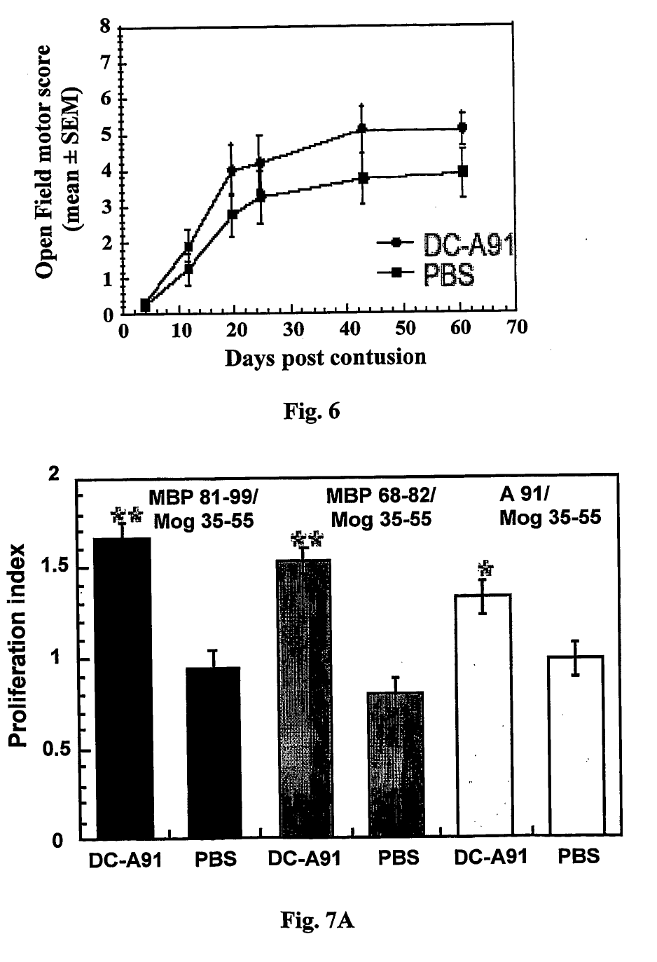

To determine whether the observed neuroprotective effect of the treatment with DCs is T cell-dependent, we injected MBP-A91-pulsed DCs locally into spinally injured adult male SPD rats that had been thymectomized at birth and therefore lacked mature T cells. In the absence of normal T cell function, MBP-A91-pulsed DCs had no significant effect on finctional recovery (FIG. 6). The results shown are of one representative experiment of three experiments carried out using thymectomized SPD rats; similar results were obtained in males and females. It should be noted that due to variations in animal weight from one experiment to another,r we always compared BBB scores among groups within the same experiments. Thus the relative high BBB score in the control group of the thymectomized animals should not be taken as an argument for the failure of the DCs in these animals to promote recovery. The lack of a T cell-mediated response in thymectomized rats was confirmed by injecting the rats with bovine serum albumin in adjuvant and assessing their ex vivo splenocyte proliferative response to the vaccination (data not shown).

We also examined whether DCs activated by lipopolysaccharide (LPS), a glycolipid component of cell wall of gram-negative bacteria that plays a role in infection-associated inflammation (Galanos and Freudenberg, 1993; Ulevitch and Tobias, 1994), would improve functional recovery by inducing a more vigorous immune response. The results showed that incubation of DCs with LPS failed to induce a significant beneficial effect. Moreover, DCs pulsed with both LPS and MBP-A91 showed the same effect as DCs pulsed with MBP-A91 only (data not shown). These results suggest that the local immune response needed for spinal cord recovery is antigen specific.

### Example 4

Systemic Administration of MBP-A91-Pulsed Dendritic Cells Promotes Functional Recovery

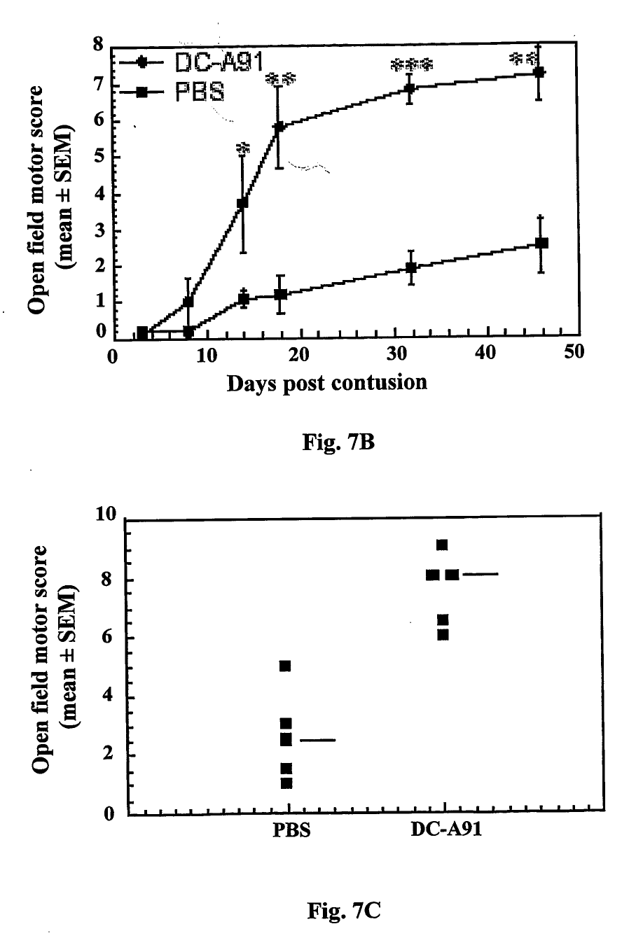

Since the DCs were found to be mature and their mechanism of action T cell-dependent, it was of interest to determine whether their beneficial effect on recovery could be reproduced by their systemic administration. We first examined whether systemic injection of pulsed DCs can evoke a systemic T cell response specific to the pulsing antigen. Spinally injured SPD males were injected i.v. with 1×10⁶ MBP-A91-pulsed DCs or with PBS (FIG. 7). Ten days later, three rats from each group were euthanized, their spleens were removed, and splenocyte proliferation was assayed in the presence of different myelin peptides. FIG. 7A depicts the proliferation of splenocytes in the presence of each of the tested peptides (MBP-A91, MBP 81-99, and MBP 68-82) relative to their proliferation in the presence of a control myelin-derived peptide MOG 35-55. Splenocytes from rats injected i.v. with MBP-A91-pulsed DCs, but not from rats injected with PBS, displayed a significantly stronger T cell response to the MBP peptides than to the MOG peptide. These results suggest that systemic administration of 1×10⁶ DCs pulsed with MBP-A91 evokes a T cell response to the directly related MBP peptide A91, as well as to other MBP-derived peptides such as MBP 81-99 and, possibly through a mechanism of epitope spreading, alsolso to MBP 68-82 (Wildbaum, et al., 2002).

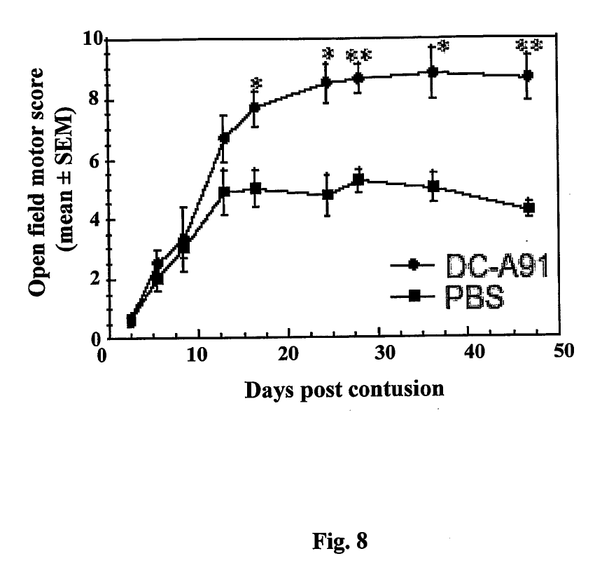

These findings encouraged us to examine the effect of this systemic route of administration on the finctional recovery of spinally injured rats. Male SPD rats were subjected to SCI and immediately afterwards received MBP-A91-pulsed DCs by i.v. injection. Starting 15 days after the injury, a significant effect on recovery was observed at all time points tested (FIGS. 7B, 7C). The highest score attained by any rat was 9.5, which was not significantly higher than that of rats injected via other routes; however, the number of rats that recovered after i.v. injection was higher than that observed after treatment via other routes. Interestingly, in spinally injured SPD rats, administration of MBP-A91-pulsed DCs (2×10⁶ cells/rat) by s.c. injection also led to significantly better recovery than that obtained in PBS-injected matched controls (two-factor repeated measures ANOVA, P≦0.01, df=1, F-test=12.353) (FIG. 8). Note that the SPD rats used in this experiment were females, and therefore their spontaneous recovery was better than that of males (FIG. 8).

### Example 5

Therapeutic Window of Dendritic Vaccination after Spinal Cord Contusion

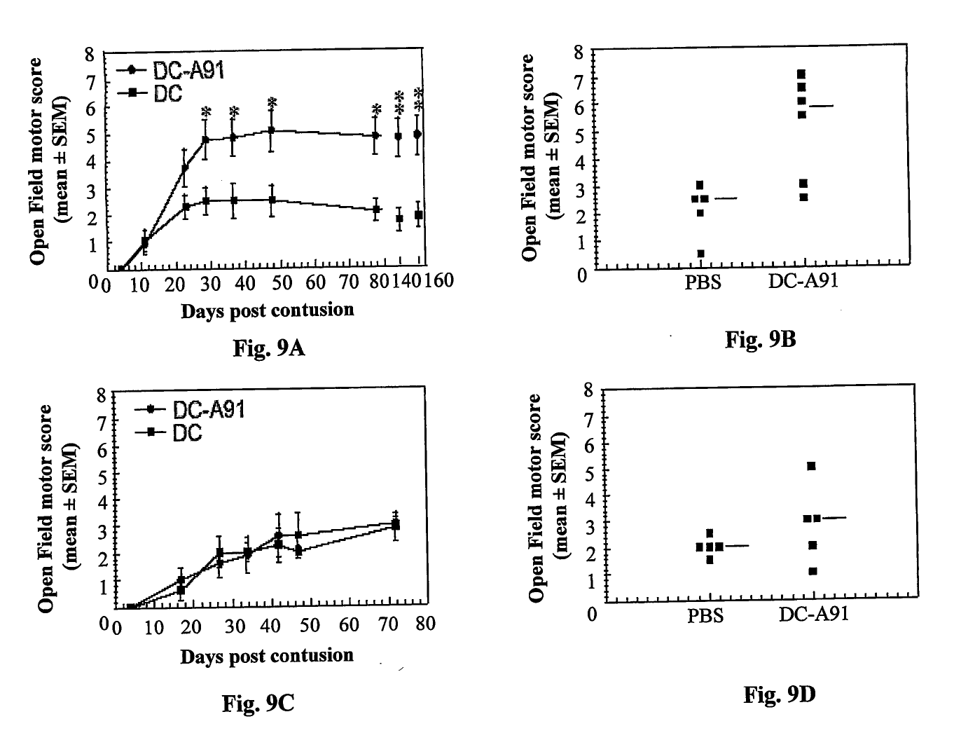

To examine the effect of delayed local injection of MBP-A91-pulsed DCs, 20 SPD male rats underwent severe spinal cord contusion and their BBB locomotor scores were monitored during the first 10 days after injury. On the 11^{th} day, the 12 rats with the lowest BBB scores were randomly assigned to two groups, whose mean BBB scores±SEM were similar (1.0±0.4 in group 1 and 0.9±0.25 in group 2; P≦0.87, two-tailed Student's t-test). One day later (12 days after SCI), the lesion site was injected with 5×10⁵ MBP-A91-pulsed DCs in one group of rats and with non-pulsed DCs in the other. Starting from day 29 after injury, locomotor (BBB) scores in the two groups of rats were found to differ significantly (P≦0.05, two-tailed Student's t-test; FIGS. 9A, 9B). The overall effect of the delayed treatment with MBP-A91-pulsed DCs on functional recovery was statistically significant (two-factor repeated measures ANOVA, P≦0.05, df=1, F-test=6.206). When this experimental paradigm was repeated, with local administration of MBP-A91-pulsed DCs performed 28 days after the injury, the DCs had no significant effect on locomotor recovery (FIGS. 9C, 9D).

### Example 6

Morphological Evidence of Improved Preservation of Neural Tissue After Vaccination with Dendritic Cells Pulsed with MBP Peptide

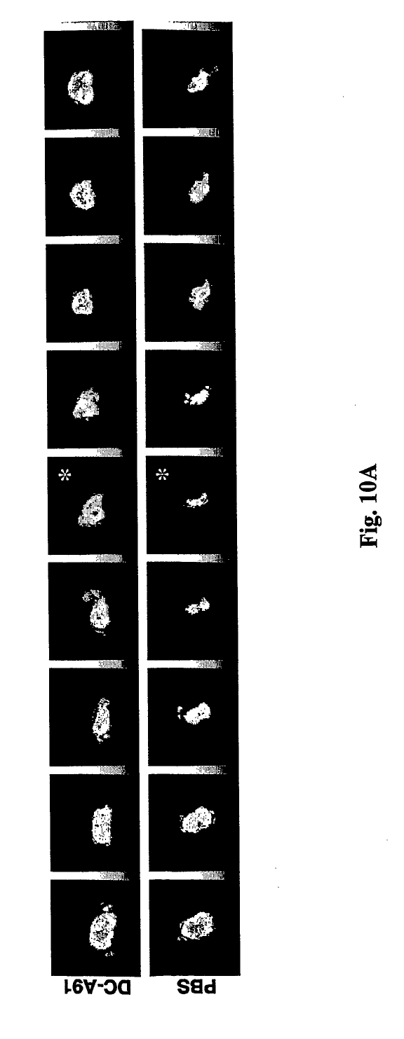

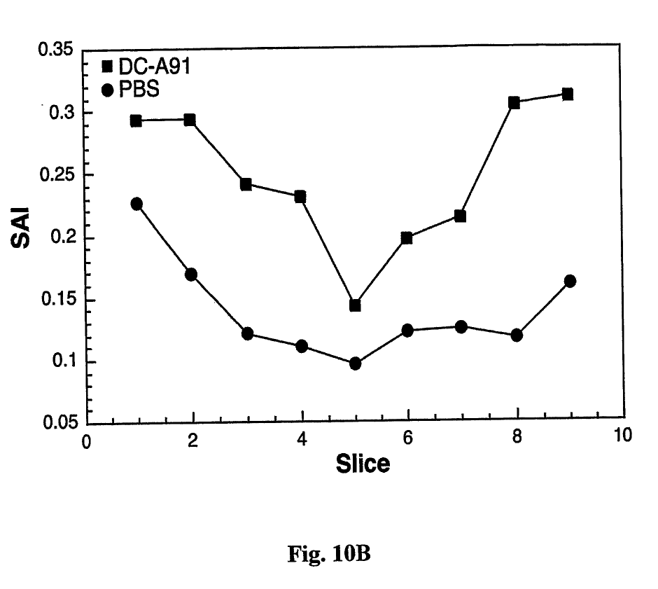

Nine months after contusive SCI that was immediately followed by local injection of MBP-A91-pulsed DCs or PBS into the site of injury, a spinal cord segment (approximately 3 cm, with the site of injury in the middle) was excised from two euthenized male SPD rats in each group and scanned by diffusion-weighted MRI (DW-MRI). Virtual slices of 0.5 mm were analyzed at intervals of 1.18 mm. The acquired axial images were analyzed to yield the apparent diffusion coefficient values and the values of anisotropy of the tissue—a marker of white matter integrity (Nevo, et al., 2001). The axial anisotropy maps derived from the DW-MRI images present consecutive areas of diffusion anisotropy along the excised cord. In the maps of the group treated with MBP-A91-pulsed DCs, the area of anisotropy is wider than in the PBS-treated controls (FIG. 10A). In contrast to the continuous longitudinal structure seen in the treated rats, slices taken from the PBS-injected controls show a loss of organized structure at the center of the lesion site, and the area of diffusion anisotropy is relatively small even in slices distant from the site of lesion (FIG. 10A). Moreover, quantitative analysis showed that the sum of anisotropy (SAI)—representing the integrated anisotropy value—was higher in the rats treated with MBP-A91-pulsed DCs than in the controls throughout the entire length of scanned segments (FIG. 10B). The behavioral outcome correlated well with the MRI results: the higher the behavioral score, the larger the area of diffusion anisotropy found at the site of the lesion (Hauben, et al., 2000).

Discussion

The results presented above show a significant improvement in locomotor function after contusive SCI in rats treated by local or systemic injection of bone marrow-derived DCs pulsed in vitro with MBP-derived or related peptides. The beneficial effect of the treatment was also evident morphologically, with better preservation of neural tissue seen on histological examination and a decrease in the size of cavities in the spinal cords of treated rats examined by MRI.

Contrary to the prevailing assumption, DCs were found to populate specific areas of the CNS including the meninges and choroid plexus (McMenamin, 1999). This presumably enables DCs to “sample” the CNS environment and present CNS antigens in the lymph nodes. In other organs and tissues, DCs under physiological conditions present self-antigens in the context of MHC but lack the costimulatory capacity to initiate a response against them. This may well hold true also for CNS antigens, and might contribute to the maintenance of peripheral tolerance to CNS-associated self-antigens. Under pathological conditions, for example after injury to cells, DCs undergo a process of maturation that enables them to present tissue-derived antigens to T lymphocytes in a highly efficient and stimulatory way. After ischemic injury to the CNS in mature rats DCs accumulate at the lesion site (Kostulas, et al., 2002), and contusive SCI in rats is followed by upregulated expression of chemoattractants of DCs (McTigue, et al., 1998). The present inventors have shown that stimulation of an adaptive immune response against CNS self-antigens after an injury is a normal part of the body's own healing mechanism (Yoles, et al., 2001), and a central feature of a proposed new concept of “protective autoimmunity” (Moalem, et al., 1999; Schwartz, et al., 1999).

The endogenous autoimmune response, though apparently sufficient for day-to-day maintenance (Nevo, et al., 2003; Schwartz and Kipnis, 2002), seems to be insufficient to arrest the secondary degeneration accompanying CNS trauma; it can, however, be boosted by innate and adaptive immune manipulations (Fisher, et al., 2001; Hauben, et al., 2000; Moalem, et al., 1999). DCs have been shown to initiate specific immune responses. Such manipulations have been utilized to evoke immune responses to tumor-associated antigens, and are currently undergoing clinical testing for the treatment of cancer (Lau, et al., 2001).

A number of studies have shown that DCs can induce immunological tolerance and prevent development of EAE. The DCs used in all of those studies were probably immature or semi-mature (Lutz and Schuler, 2002). By using surface markers and specific cytokines, we showed that the DCs used in the present application were mature. Moreover, tolerance induction by our DCs was ruled out by the observation that the ex-vivo proliferation of splenocytes from rats treated with DCs pulsed with MBP related peptides was enhanced in response to MBP peptides. This conclusion is in line with recent results from our laboratory demonstrating that the outcome of CNS injury in rats is worsened if the animals were neonatally tolerized to myelin-associated antigens or if they received regulatory CD4⁺CD25⁺ T cells that suppress the ability to manifest an immune response (Kipnis, et al., 2002; Schwartz and Kipnis, 2002).

Despite the ability of DCs to stimulate an effective immune response, immune activation by DCs pulsed with specific encephalitogenic self-antigens in normal rats or mice has never, to our knowledge, been shown to induce EAE. In one study EAE was induced in irradiated mice by administration of DCs, but only when CD4⁺ T cells specific to an encephalitogenic peptide of MBP were administered at the same time (Dittel, et al., 1999). In our laboratory, no symptoms of EAE were observed after the antigen-pulsed DCs that improved recovery from SCI in this study were injected, either locally (in the spinal cord), subcutaneously, or intravenously into naïve rats (data not presented). Treatment with DCs in this way thus appears to be safe, insofar as it evokes a desired immune response while avoiding destructive autoimmunity.

The experiments in the present application were conducted in two rat strains, SPD and Lewis, that differ in their resistance to EAE and in their ability to withstand CNS trauma (Kipnis, et al., 2001). In both strains the treatment with antigen-pulsed DCs was effective. In the Lewis strain we used female rats, in which spontaneous recovery (measured by the BBB score) is evidently superior to that in male rats (Hauben, et al., 2002; Stein, 2001). Nevertheless, DC treatment was still effective in the females, suggesting that the female endogenous response counteracts some but not all of the injury-induced degeneration.

We showed here that recovery from SCI is improved even if the DC injection is delayed for as long as 12 days after the injury. The locomotor activity of the rats used in that experiment was assessed prior to treatment, and only rats with a BBB score lower than 2 were used. This homogeneous group of rats was randomly divided into two groups, one of which was treated with the DCs and the other served as a control. This approach minimizes variations in insult severity among the contused rats within the same groups and between the treated and non-reated group. Interestingly, only a slight improvement was seen in the control group, whereas the outcome in the treated rats was almost as good as if they had been treated immediately after the injury. This finding might suggest that the mechanisms underlying functional recovery in DC-treated rats involve both neuroprotection and sprouting. Neuroprotection is limited by its operation only during the time period in which axons have not yet degenerated, whereas sprouting may make a significant contribution later. The relatively wide therapeutic time window may be of clinical importance in the treatment of patients with SCI. While bearing in mind previous findings in connection with regeneration and functional recovery induced by implantation of macrophages into transected spinal cords of rats (Rapalino, et al., 1998), and the fact that passive or active immunization with myelin peptides causes local activation of macrophages and microglia (Butovsky, et al., 2001), the possibility remains that DC injection, in addition to its neuroprotective effect on uninjured axons, causes sprouting and regeneration of injured axons.

Treatment with non-pulsed DCs or with DCs pulsed with the irrelevant protein ovalbumin had no effect on the injured spinal cord. DCs pulsed with MBP peptide or the altered MBP peptide A91 were beneficial, indicating that mature DCs act as antigen-presenting cells and can therefore be active only in conjunction with the relevant CNS antigen when administered to spinally injured rats.

In one preferred embodiment of the present invention, the altered MBP peptide A91 was used instead of natural peptides of MBP because the altered peptide, though as effective as MBP as a vaccine for SCI, is not encephalitogenic (Hauben, et al., 2001b). There was no difference in phenotype between pulsed and non-pulsed DCs, as indicated by the identical expression of cytokines in both. One should bear in mind that both pulsed DCs and the control non-pulsed DCs were exposed to many other irrelevant proteins during their growth in a serum-rich medium (10% fetal calf serum), as well as in the last few hours of pulsing when the specific peptide was added. These facts lend even more support to the contention that the treatment is antigen specific. Moreover, stimulation of the DCs with LPS, a strong pro-inflammatory compound, did not substitute the need for the antigen specific pulsing.

Several finding in the present application suggest that the effect of DCs on the injured spinal cord is mediated via systemic immune mechanisms. First, there were no significant differences between the effects of DCs administered locally, subcutaneously, or intravenously to the spinal cord. Second, splenocytes from treated rats, when stimulated ex vivo with different antigens, showed a stronger proliferative response to myelin peptides than did splenocytes from untreated rats. This clearly indicates that the DCs administered to the contused rats evoked a systemic immune response to myelin peptides. The DCs administered in that experiment were pulsed with the altered myelin peptide A91 (the peptide used for pulsing of DCs in the behavioral experiments), and probably evoked a response to the ex vivo-tested dominant myelin peptides because of antigenic similarity and epitope spreading. Third, when injected into rats that were thymectomized at birth, DCs had no effect on recovery from contusive SCI. Neonatally thymectomized rats are devoid of mature T lymphocytes (which normally develop in the thymus of the newborn), indicating that the beneficial effect of DCs on spinal cord recovery is at least partly dependent on T cells. The injected DCs thus evoke a systemic, antigen-specific, T cell-dependent immune response.

Although, as mentioned above, different routes of DC administration did not significantly affect the maximal recovery achieved, a high BBB score was achieved by more rats when the treatment was administered intravenously. This might reflect the uniformity of intravenous treatment relative to the subcutaneous or local administration of DCs. It is reasonable to assume that the intravenously injected DCs reach the spleen and other lymphoid organs. Intravenously injected mature DCs have been shown to reach the spleen within 1 day of injection and to localize preferentially in the T cell area of the spleen (Sai, et al., 2002). Intravenous route for DC administration is known to be effective both for induction of immune tolerance (Menges, et al., 2002) and for induction of immune activation (Fong, et al., 2001; Lau, et al., 2001; Sai, et al., 2002).

The results herein clearly show a beneficial effect of DCs pulsed with specific antigens on recovery of rats from SCI. We conclude that the effect of DCs is systemic and T cell dependent, and that—similarly to vaccination with myelin peptides—it evokes an adaptive immune response directed to peptides of MBP. Our treatment can therefore be viewed as a DC vaccination and, like vaccination with the peptide in adjuvant, is a means of helping the local innate response to cope with stressful injury-induced conditions by promoting the body's own self-repair mechanism, namely the adaptive systemic immune response against antigens residing in the lesion site.

### REFERENCES

Banchereau and Steinman (1998) Dendritic cells and the control of immunity, Nature 392:245-252.

Basso, D M, Beattie, M S, Bresnahan, J C (1996) Graded histological and locomotor outcomes after spinal cord contusion using the NYU weight-drop device versus transection. Exp Neurol 139:244-256.

Bavetta, S, Hamlyn, P J, Burnstock, G, Lieberman, A R, Anderson, P N (1999) The effects of FK506 on dorsal column axons following spinal cord injury in adult rats: neuroprotection and local regeneration. Exp Neurol 158:382-393.

Bazan, N G, Rodriguez de Turco, E B, Allan, G (1995) Mediators of injury in neurotrauma: intracellular signal transduction and gene expression. J Neurotrauma 12:791-814.

Beattie, M S, Bresnahan, J C, Komon, J, Tovar, C A, Van Meter, M, Anderson, D K, Faden, A I, Hsu, C Y, Noble, L J, Salzman, S, Young, W (1997) Endogenous repair after spinal cord contusion injuries in the rat. Exp Neurol 148:453-463.

Behrmann, D L, Bresnahan, J C, Beattie, M S (1994) Modeling of acute spinal cord injury in the rat: neuroprotection and enhanced recovery with methylprednisolone, U-74006F and YM-14673. Exp Neurol 126:61-75.

Bethea, J R, Nagashima, H, Acosta, M C, Briceno, C, Gomez, F, Marcillo, A E, Loor, K, Green, J, Dietrich, W D (1999) Systemically administered interleukin-10 reduces tumor necrosis factor-alpha production and significantly improves functional recovery following traumatic spinal cord injury in rats. J Neurotrauma 16:851-863.

Blesch, A, Tuszynski, M H (1997) Robust growth of chronically injured spinal cord axons induced by grafts of genetically modified NGF-secreting cells. Exp Neurol 148:444-452.

Bregman, B S (1998) Regeneration in the spinal cord. Curr Opin Neurobiol 8:800-807.

Brewer, K L, Bethea, J R, Yezierski, R P (1999) Neuroprotective effects of interleukin-10 following excitotoxic spinal cord injury. Exp Neurol 159:484-493.

Butovsky, 0, Hauben, E, Schwartz, M (2001) Morphological aspects of spinal cord autoimmune neuroprotection: colocalization of T cells with B7-2 (CD86) and prevention of cyst formation. Faseb J 15:1065-1067.

Caux et al. (1996) CD34⁺ hematopoietic progenitors from human cord blood differentiate along two independent pathways in response to GM-CSF and TNF-α, J. Exp. Med. 184:695-706.

Caux et al. (1997) CD34⁺ hematopoietic progenitors from human cord blood differentiate along two independent dendritic cell pathways in response to granulocyte-macrophage colony-stimulating factor plus tumor necrosis factor alpha. II. Functional analysis. Blood 90:1458-1470.

Constantini, S, Young, W (1994) The effects of methylprednisolone and the ganglioside GM1 on acute spinal cord injury in rats. J Neurosurg 80:97-111.

Crowe, M J, Bresnahan, J C, Shuman, S L, Masters, J N, Beattie, M S (1997) Apoptosis and delayed degeneration after spinal cord injury in rats and monkeys. Nat Med 3:73-76.

Dittel, B N, Visintin, I, Merchant, R M, Janeway, C A, Jr. (1999) Presentation of the self antigen myelin basic protein by dendritic cells leads to experimental autoimmune encephalomyelitis. J Immmunol 163:32-39.

Faden, A I (1993) Experimental neurobiology of central nervous system trauma. Crit Rev Neurobiol 7:175-186.

Fisher, J, Levkovitch-Verbin, H, Schori, H, Yoles, E, Butovsky, O, Kaye, J F, Ben-Nun, A, Schwartz, M (2001) Vaccination for neuroprotection in the mouse optic nerve: implications for optic neuropathies. J Neurosci 21:136-142.

Fong, L, Brockstedt, D, Benike, C, Wu, L, Engleman, E G (2001) Dendritic cells injected via different routes induce immunity in cancer patients. J Immunol 166:4254-4259.

Franzen, R, Schoenen, J, Leprince, P, Joosten, E, Moonen, G, Martin, D (1998) Effects of macrophage transplantation in the injured adult rat spinal cord: a combined immunocytochemical and biochemical study. J Neurosci Res 51:316-327.

Galanos, C, Freudenberg, M A (1993) Mechanisms of endotoxin shock and endotoxin hypersensitivity. Immunobiology 187:346-356.

Garderet L, Cao H, Salarnero J, Verge V, Tisserand E, Scholl S, Gorin N C, Lopez M. (2001) In vitro production of dendritic cells from human blood monocytes for therapeutic use. J. Hematother. Stem. Cell Res. 10(4):553-567

Gaur, A, Boehme, S A, Chalmers, D, Crowe, P D, Pahuja, A, Ling, N, Brocke, S, Steinman, L, Conlon, P J (1997) Amelioration of relapsing experimental autoimmune encephalomyelitis with altered myelin basic protein peptides involves different cellular mechanisms. J Neuroimmunol 74:149-158.

Geijtenbeek et al, “DC-SIGN, a dendritic cell-specific HIV-1-binding protein that enhances trans-infection of T cells”, Cell 100:587-597 (2000)

Guyre et al. (2002) Advantages of hydrophobic culture bags over flasks for the generation of monocyte-derived dendritic cells for clinical applications. J. Immunol. Methods 262(1-2):85-94.

Hauben, E, Butovsky, O, Nevo, U, Yoles, E, Moalem, G, Agranov, E, Mor, F, Leibowitz-Amit, R, Pevsner, E, Akselrod, S, Neeman, M, Cohen, I R, Schwartz, M (2000) Passive or active immunization with myelin basic protein promotes recovery from spinal cord contusion. J Neurosci 20:6421-6430.

Hauben, E, Ibarra, A, Mizrahi, T, Barouch, R, Agranov, E, Schwartz, M (2001a) Vaccination with a Nogo-A-derived peptide after incomplete spinal-cord injury promotes recovery via a T-cell-mediated neuroprotective response: comparison with other myelin antigens. Proc Natl Acad Sci U S A 98:15173-15178.

Hauben, E, Agranov, E, Gothilf, A, Nevo, U, Cohen, A, Smirnov, I, Steinman, L, Schwartz, M (2001b) Posttraumatic therapeutic vaccination with modified myelin self-antigen prevents complete paralysis while avoiding autoimmune disease. J Clin Invest 108:591-599.

Hauben, E, Mizrahi, T, Agranov, E, Schwartz, M (2002) Sexual dimorphism in the spontaneous recovery from spinal cord injury: a gender gap in beneficial autoimmunity? Eur J Neurosci 16:1731-1740.

Hauben, E, Nevo, U, Yoles, E, Moalem, G, Agranov, E, Mor, F, Akselrod, S, Neeman, M, Cohen, I R, Schwartz, M (2000) Autoimmune T cells as potential neuroprotective therapy for spinal cord injury. Lancet 355:286-287.

Hirschberg D L, Moalem G, He J, Mor F, Cohen I R, Schwartz M.(1998) Accumulation of passively transferred primed T cells independently of their antigen specificity following central nervous system trauma. J. Neuroimmunol. 89:88-96.

Ho et al. (2002) Spontaneous generation and survival of blood dendritic cells in mononuclear cell culture without exogenous cytokines. Blood 99(8):2897-2904.

Jakeman, L B, Guan, Z, Wei, P, Ponnappan, R, Dzwonczyk, R, Popovich, P G, Stokes, B T (2000) Traumatic spinal cord injury produced by controlled contusion in mouse. J Neurotrauma 17:299-319.

Kipnis, J, Mizrahi, T, Hauben, E, Shaked, I, Shevach, E, Schwartz, M (2002) Neuroprotective autoimmunity: Naturally occurring CD4+CD25+ regulatory T cells suppress the ability to withstand injury to the central nervous system. Proc Natl Acad Sci U S A 99:15620-15625.

Kipnis, J, Yoles, E, Schori, H, Hauben, E, Shaked, I, Schwartz, M (2001) Neuronal survival after CNS insult is determined by a genetically encoded autoimmune response. J Neurosci 21:4564-4571.

Knight, S C, Burke, F, Bedford, P A (2002) Dendritic cells, antigen distribution and the initiation of primary immune responses to self and non-self antigens. Semin Cancer Biol 12:301-308.

Kostulas, N, Li, H L, Xiao, B G, Huang, Y M, Kostulas, V, Link, H (2002) Dendritic cells are present in ischemic brain after permanent middle cerebral artery occlusion in the rat. Stroke 33:1129-1134.

Lau, R, Wang, F, Jeffery, G, Marty, V, Kuniyoshi, J, Bade, E, Ryback, M E, Weber, J (2001) Phase I trial of intravenous peptide-pulsed dendritic cells in patients with metastatic melanoma. J Immunother 24:66-78.

Lazarov-Spiegler, O, Solomon, A S, Zeev-Brann, A B, Hirschberg, D L, Lavie, V, Schwartz, M (1996) Transplantation of activated macrophages overcomes central nervous system regrowth failure. Faseb J 10:1296-1302.

Link, H, Huang, Y M, Masterman, T, Xiao, B G (2001) Vaccination with autologous dendritic cells: from experimental autoimmune encephalomyelitis to multiple sclerosis. J Neuroimmunol 114:1-7.

Liu, D, Yang, R, Yan, X, McAdoo, D J (1994) Hydroxyl radicals generated in vivo kill neurons in the rat spinal cord: electrophysiological, histological, and neurochemical results. J Neurochem 62:37-44.

Liu, Y J (2001) Dendritic cell subsets and lineages, and their functions in innate and adaptive immunity. Cell 106:259-262.

Lutz, M, Schuler, G (2002) Immature, semi-mature and fully mature dendritic cells: which signals induce tolerance or immunity? Trends Immunol 23:445.

Lutz, M B, Kukutsch, N, Ogilvie, A L, Rossner, S, Koch, F, Romani, N, Schuler, G (1999) An advanced culture method for generating large quantities of highly pure dendritic cells from mouse bone marrow. J Immunol Methods 223:77-92.

Ma, M, Basso, D M, Walters, P, Stokes, B T, Jakeman, L B (2001) Behavioral and histological outcomes following graded spinal cord contusion injury in the C57B1/6 mouse. Exp Neurol 169:239-254.

McMenamin, P G (1999) Distribution and phenotype of dendritic cells and resident tissue macrophages in the dura mater, leptomeninges, and choroid plexus of the rat brain as demonstrated in wholemount preparations. J Comp Neurol 405:553-562.

McTigue, D M, Tani, M, Krivacic, K, Chemosky, A, Kelner, G S, Maciejewski, D, Maki, R, Ransohoff, R M, Stokes, B T (1998) Selective chemokine mRNA accumulation in the rat spinal cord after contusion injury. J Neurosci Res 53:368-376.

Mellman, I, Steinman, R M (2001) Dendritic cells: specialized and regulated antigen processing machines. Cell 106:255-258.

Menges, M, Rossner, S, Voigtlander, C, Schindler, H, Kukutsch, N A, Bogdan, C, Erb, K, Schuler, G, Lutz, M B (2002) Repetitive injections of dendritic cells matured with tumor necrosis factor alpha induce antigen-specific protection of mice from autoimmunity. J Exp Med 195:15-21.

Moalem, G, Leibowitz-Amit, R, Yoles, E, Mor, F, Cohen, I R, Schwartz, M (1999) Autoimmune T cells protect neurons from secondary degeneration after central nervous system axotomy. Nat Med 5:49-55.

Nevo, U, Hauben, E, Yoles, E, Agranov, E, Akselrod, S, Schwartz, M, Neeman, M (2001) Diffusion anisotropy MRI for quantitative assessment of recovery in injured rat spinal cord. Magn Reson Med 45:1-9.

Nevo, U, Kipnis, J, Golding, I, Shaked, I, Neumann, A, Akselrod, S, Schwartz, M (2003) Autoimmune as a special case of immunity: removing threats from within. Trends Mol Med 9:88-93.

Pierre et al. (1997) Developmental regulation of MHC class II transport in mouse dendritic cells. Nature 388:787-792.

Rapalino, O, Lazarov-Spiegler, O, Agranov, E, Velan, G J, Yoles, E, Fraidakis, M, Solomon, A, Gepstein, R, Katz, A, Belkin, M, Hadani, M, Schwartz, M (1998) Implantation of stimulated homologous macrophages results in partial recovery of paraplegic rats. Nat Med 4:814-821.

Sai, T, Milling, S W, Mintz, B (2002) Freezing and thawing of bone marrow-derived murine dendritic cells with subsequent retention of immunophenotype and of antigen processing and presentation characteristics. J Immunol Methods 264:153-162.

Sallusto and Lanzavecchia (1994) Efficient presentation of soluble antigen by cultured human dendritic cells is maintained by granulocyte/macrophage colony-stimulating factor plus interleukin 4 and down regulated by tumor necrosis factor alpha. J. Exp. Med. 179:1109-1118.

Schwartz, M, Kipnis, J (2002) Autoimmunity on alert: naturally occurring regulatory CD4(+)CD25(+) T cells as part of the evolutionary compromise between a ‘need’ and a ‘risk’. Trends Immunol 23:530-534.

Schwartz, M, Moalem, G, Leibowitz-Amit, R, Cohen, I R (1999) Innate and adaptive immune responses can be beneficial for CNS repair. Trends Neurosci 22:295-299.

Stein, D G (2001) Brain damage, sex hormones and recovery: a new role for progesterone and estrogen? Trends Neurosci 24:386-391.

Talmor, M, Mirza, A, Turley, S, Mellman, I, Hoffinan, L A, Steinman, R M (1998) Generation or large numbers of immature and mature dendritic cells from rat bone marrow cultures. Eur J Immunol 28:811-817.

Turley, S J (2002) Dendritic cells: inciting and inhibiting autoimmunity. Curr Opin Immunol 14:765-770.

Ulevitch, R J, Tobias, P S (1994) Recognition of endotoxin by cells leading to transmembrane signaling. Curr Opin Immunol 6:125-130.

Wildbaum, G, Netzer, N, Karin, N (2002) Trl cell-dependent active tolerance blunts the pathogenic effects of determinant spreading. J Clin Invest 110:701-710.

Xiao, B G, Huang, Y M, Yang, J S, Xu, L Y, Link, H (2001) Bone marrow-derived dendritic cells from experimental allergic encephalomyelitis induce immune tolerance to EAE in Lewis rats. Clin Exp Immunol 125:300-309.

Yoles, E, Hauben, E, Palgi, O, Agranov, E, Gothilf, A, Cohen, A, Kuchroo, V, Cohen, I R, Weiner, H, Schwartz, M (2001) Protective autoimmunity is a physiological response to CNS trauma. J Neurosci 21:3740-3748.

Young, W (1996) Spinal cord regeneration [comment]. Science 273:451.

1-22. (canceled)

23. A method for preventing or inhibiting neuronal degeneration, or for promoting nerve regeneration, in the central nervous system (CNS) or peripheral nervous system (PNS), which comprises administering to an individual in need thereof an effective amount of antigen-presenting cells that have been pulsed with an agent selected from the group consisting of:
(a) a nervous system (NS)-specific antigen or an analog thereof;
(b) a peptide derived from an NS specific antigen or from an analog thereof, or an analog or derivative of said peptide;
(c) a copolymer selected from the group consisting of Copolymer 1, a Copolymer 1-related peptide or polypeptide, and poly-Glu⁵⁰Tyr⁵⁰; and
(d) a non-self antigen.

24. A method according to claim 23, wherein said antigen-presenting cells are human antigen-presenting cells.

25. A method according to claim 24, wherein said antigen-presenting cells are selected from the group consisting of monocytes, macrophages, dendritic cells and B cells.

26. A method according to claim 25, wherein said antigen-presenting cells are autologous dendritic cells obtained from the individual in need.

27. The method according to claim 26, in which the dendritic cells were obtained from skin, spleen, thymus, bone marrow, lymph nodes or peripheral blood of said individual.

28. A method according to claim 23, wherein said antigen-presenting cells have been cultured in a medium containing at least one stimulatory biologically active agent selected from the group consisting of transforming growth factor-beta (TGF-β), β-interferon (IFN-β), IFN-γ, tumor necrosis factor-α (TNF-α), interleukin 2 (IL-2), IL-3, IL-4, IL-6, IL-10, monocyte chemotactic and activating factor (MCAF), granulocyte colony stimulating factor (G-CSF), macrophage colony stimulating factor (M-CSF), granulocyte-macrophage colony stimulating factor (GM-CSF), colony stimulating factor 1 (CSF-1), neurotrophic factor 3 (NT-3), nerve growth factor (NGF), brain-derived neurotrophic factor (BDNF), lipid A, the tripeptide fMet-Leu-Phe (Fmlp), muramyl dipeptide (MDP), the ionophore A23187, vitamin D3-binding protein, CD40 ligand and lipopolysaccharide (LPS).

29. A method according to claim 28, wherein said antigen-presenting cells have been cultured in a medium containing IL-4, GM-CSF, or both IL-4 and GM-CSF.

30. The method according to claim 29, wherein said antigen-presenting cells are human dendritic cells that have been cultured in a medium containing both IL-4 and GM-CSF.

31. A method for treatment of an injury, disorder or disease of the CNS or PNS, which comprises administering to an individual in need thereof an effective amount of antigen-presenting cells that have been pulsed with an agent selected from the group consisting of:
(a) a nervous system (NS)-specific antigen or an analog thereof;
(b) a peptide derived from an NS-specific antigen or from an analog thereof, or an analog or derivative of said peptide;
(c) a copolymer selected from the group consisting of Copolymer 1, a Copolymer 1-related peptide or polypeptide, and poly-Glu⁵⁰Tyr⁵⁰; and
(d) a non-self antigen.

32. A method according to claim 31, wherein the injury in the CNS is spinal cord injury, blunt trauma, penetrating trauma, brain coup or contrecoup, hemorrhagic stroke, or ischemic stroke.

33. A method according to claim 31, wherein the disorder or disease is diabetic neuropathy, senile dementia, Alzheimer's disease, Parkinson's disease, facial nerve (Bell's) palsy, Huntington's chorea, amyotrophic lateral sclerosis (ALS), vitamin deficiency, epilepsy, amnesia, anxiety, hyperalgesia, psychosis, seizures, oxidative stress, opiate tolerance and dependence, glaucoma, optic neuropathy, age-related macular degeneration or retinal degeneration.

34. A method according to claim 31 wherein said antigen presenting cells are administered locally at or near the site of injury,

35. A method according to claim 31 wherein said antigen presenting cells are administered sistemically

36. A method for treatment of spinal cord injury, which comprises administering to an individual in need thereof an effective amount of autologous dendritic cells that have been pulsed with an agent selected from the group consisting of:
(a) a nervous system (NS)-specific antigen or an analog thereof;
(b) a peptide derived from an NS-specific antigen or from an analog thereof, or an analog or derivative of said peptide;
(c) a copolymer selected from the group consisting of Copolymer 1, a Copolymer 1-related peptide or polypeptide, and poly-Glu⁵⁰Tyr⁵⁰; and
(d) a non-self antigen.

37. The method according to claim 36 wherein said autologous dendritic cells have been cultured in a medium comprising GM-CSF and IL-4 and then pulsed with the peptide of SEQ ID NO:4.

38. A method for treatment of an injury of the CNS or PNS, which comprises immunizing an individual in need thereof with a non-self-antigen and thereafter administering to said individual at the injury site an effective amount of antigen-presenting cells that have been pulsed with said non-self antigen.

39. A method for treatment of an injury of the CNS or PNS, which comprises administering to an individual in need at the injury site an effective amount of antigen-presenting cells that have been pulsed with a non-self antigen, wherein said individual is an individual that has been exposed previously to said non-self antigen.
